SEQUENCE LISTING (78 sequences)

SEQ ID NO: 1 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 17 aa, Artificial Sequence
  Exemplary E200 epitope
GHNYTTRNILPGLNITC

SEQ ID NO: 3 - protein, 16 aa, Artificial Sequence
  Variant E200 epitope peptide (E200′)
GHNYTTRNILPGLNIT

SEQ ID NO: 4 - protein, 17 aa, Artificial Sequence
  E200 epitope Cys to Ser modification
GHNYTTRNILPGLNITS

SEQ ID NO: 5 - protein, 21 aa, Artificial Sequence
  Extended E200 Cys to Ser modification
GHNYTTRNILPGLNITSTFHK

SEQ ID NO: 6 - protein, 22 aa, Artificial Sequence
  Extended E200′ Cys to Ser modification (22 aa)
GHNYTTRNILPGLNITSTFHKT

SEQ ID NO: 7 - protein, 23 aa, Artificial Sequence
  Extended E200′′ Cys to Ser modification
GHNYTTRNILPGLNITSTFHKTC

SEQ ID NO: 8 - protein, 16 aa, Artificial Sequence
  Pep16
DFPGHNYTTRNILPGC

SEQ ID NO: 9 - protein, 23 aa, Artificial Sequence
  Pep17
GHNYTTRNILPGLNITSTFHKTS

SEQ ID NO: 10 - protein, 27 aa, Artificial Sequence
  Extended Pep17 (27 aa)
GHNYTTRNILPGLNITSTFHKTSGSGK

SEQ ID NO: 11 - protein, 11 aa, Artificial Sequence
  Minimum sequence E200 peptide (target epitope)
NYTTRNILPGL

SEQ ID NO: 12 - protein, 15 aa, Artificial Sequence
  Exemplary E300 epitope
KYYKENNVEKRTLIK

SEQ ID NO: 13 - protein, 17 aa, Artificial Sequence
  Variant E300 epitope peptide (E30′)
KYYKENNVEKRTLIKVF

SEQ ID NO: 14 - protein, 25 aa, Artificial Sequence
  Exemplary E200/E300 or composite epitope
GHNYTTRNILPGAGAKYYKENNVEK

SEQ ID NO: 15 - protein, 22 aa, Artificial Sequence
  E200 + G4S linker
GHNYTTRNILPGLNITSGGGGS

SEQ ID NO: 16 - protein, 27 aa, Artificial Sequence
  E200 + 2xG4S linker
GHNYTTRNILPGLNITSGGGGSGGGGS

SEQ ID NO: 17 - protein, 32 aa, Artificial Sequence
  E200+3xG4S linker
GHNYTTRNILPGLNITSGGGGSGGGGSGGGGS

SEQ ID NO: 18 - protein, 24 aa, Artificial Sequence
  E200_extended peptide 17v3 (24 aa)
GHNYTTRNILPGLNITSTFHKTGS

SEQ ID NO: 19 - protein, 22 aa, Artificial Sequence
  E200_extended peptide 17v4 (22 aa)
GHNYTTRNILPGLNITSTFHGS

SEQ ID NO: 20 - protein, 19 aa, Artificial Sequence
  E200_extended peptide 17v5 (19 aa)
GHNYTTRNILPGLNITSGS

SEQ ID NO: 21 - protein, 22 aa, Artificial Sequence
  E200_extended peptide 17v6 (22 aa)
DFPGHNYTTRNILPGLNITSGS

SEQ ID NO: 22 - protein, 25 aa, Artificial Sequence
  E200_extended peptide 17v7 (25 aa) + linker
DFPGHNYTTRNILPGLNITSGGGGS

SEQ ID NO: 23 - protein, 30 aa, Artificial Sequence
  E200_extended peptide 17v8 (30 aa) + linker
DFPGHNYTTRNILPGLNITSGGGGSGGGGS

SEQ ID NO: 24 - protein, 35 aa, Artificial Sequence
  E200_extended peptide 17v9 (35 aa) + linker
DFPGHNYTTRNILPGLNITSGGGGSGGGGSGGGGS

SEQ ID NO: 25 - protein, 30 aa, Artificial Sequence
  E200_extended peptide 17v10 (30 aa)
DFPGHNYTTRNILPGLNITSTFHKTSGSGK

SEQ ID NO: 26 - protein, 32 aa, Artificial Sequence
  E200_extended peptide 17v11 (32 aa) + linker
DFPGHNYTTRNILPGLNITSTFHKTSGSGKGS

SEQ ID NO: 27 - protein, 35 aa, Artificial Sequence
  E200_extended peptide 17v12 (35 aa) + linker
DFPGHNYTTRNILPGLNITSTFHKTSGSGKGGGGS

SEQ ID NO: 28 - protein, 28 aa, Artificial Sequence
  E200_extended peptide 17v13 (25 aa) + linker
DFPGHNYTTRNILPGLNITSTFHGGGGS

SEQ ID NO: 29 - protein, 25 aa, Artificial Sequence
  E200_extended peptide 17v14 (22 aa) + linker
GHNYTTRNILPGLNITSTFHGGGGS

SEQ ID NO: 30 - protein, 30 aa, Artificial Sequence
  E200_extended peptide 17v15 (30 aa) + linker
DFPGHNYTTRNILPGLNITSTFHKTGGGGS

SEQ ID NO: 31 - protein, 27 aa, Artificial Sequence
  E200_extended peptide 17v16 (27 aa) +
GHNYTTRNILPGLNITSTFHKTGGGGS

SEQ ID NO: 32 - protein, 27 aa, Artificial Sequence
  E200+IgG hinge
GHNYTTRNILPGLNITSEPKSSDKTHT

SEQ ID NO: 33 - protein, 29 aa, Artificial Sequence
  E200+GS linker_IgG hinge
GHNYTTRNILPGLNITSGSEPKSSDKTHT

SEQ ID NO: 34 - protein, 32 aa, Artificial Sequence
  E200+G4S linker+IgG hinge
GHNYTTRNILPGLNITSGGGGSEPKSSDKTHT

SEQ ID NO: 35 - protein, 37 aa, Artificial Sequence
  Extended E200+ _IgG hinge
GHNYTTRNILPGLNITSTFHKTSGSGKEPKSSDKTHT

SEQ ID NO: 36 - protein, 39 aa, Artificial Sequence
  Extended E200+ GS linker+_IgG hinge
GHNYTTRNILPGLNITSTFHKTSGSGKGSEPKSSDKTHT

SEQ ID NO: 37 - protein, 42 aa, Artificial Sequence
  Extended E200+ G4S linker+IgG hinge
GHNYTTRNILPGLNITSTFHKTSGSGKGGGGSEPKSSDKTHT

SEQ ID NO: 38 - protein, 29 aa, Artificial Sequence
  E200+IgG hinge+GS linker
GHNYTTRNILPGLNITSEPKSSDKTHTGS

SEQ ID NO: 39 - protein, 31 aa, Artificial Sequence
  E200+GS linker+IgG hinge+GS linker
GHNYTTRNILPGLNITSGSEPKSSDKTHTGS

SEQ ID NO: 40 - protein, 34 aa, Artificial Sequence
  E200+G4Slinker+IgG hinge+GS linker
GHNYTTRNILPGLNITSGGGGSEPKSSDKTHTGS

SEQ ID NO: 41 - protein, 39 aa, Artificial Sequence
  Extended E200+_IgG hinge+GSlinker
GHNYTTRNILPGLNITSTFHKTSGSGKEPKSSDKTHTGS

SEQ ID NO: 42 - protein, 41 aa, Artificial Sequence
  Extended E200+GS linker+_IgG hinge+GSlinker
GHNYTTRNILPGLNITSTFHKTSGSGKGSEPKSSDKTHTGS

SEQ ID NO: 43 - protein, 44 aa, Artificial Sequence
  Extended E200+G4S linker+_IgG hinge+GSlinker
GHNYTTRNILPGLNITSTFHKTSGSGKGGGGSEPKSSDKTHTGS

SEQ ID NO: 44 - protein, 32 aa, Artificial Sequence
  E200+IgG hinge+G4S linker
GHNYTTRNILPGLNITSEPKSSDKTHTGGGGS

SEQ ID NO: 45 - protein, 34 aa, Artificial Sequence
  E200+GS linker+IgG hinge+G4S linker
GHNYTTRNILPGLNITSGSEPKSSDKTHTGGGGS

SEQ ID NO: 46 - protein, 37 aa, Artificial Sequence
  E200+G4S linker+IgG hinge+G4S linker
GHNYTTRNILPGLNITSGGGGSEPKSSDKTHTGGGGS

SEQ ID NO: 47 - protein, 42 aa, Artificial Sequence
  Extended E200+IgG hinge+G4S linker
GHNYTTRNILPGLNITSTFHKTSGSGKEPKSSDKTHTGGGGS

SEQ ID NO: 48 - protein, 44 aa, Artificial Sequence
  Extended E200+GS linker+IgG hinge+G4S linker
GHNYTTRNILPGLNITSTFHKTSGSGKGSEPKSSDKTHTGGGGS

SEQ ID NO: 49 - protein, 47 aa, Artificial Sequence
  Extended E200+G4S linker+IgG hinge+G4S linker
GHNYTTRNILPGLNITSTFHKTSGSGKGGGGSEPKSSDKTHTGGGGS

SEQ ID NO: 50 - protein, 30 aa, Artificial Sequence
  N-term extended E200 +IgG hinge
DFPGHNYTTRNILPGLNITSEPKSSDKTHT

SEQ ID NO: 51 - protein, 32 aa, Artificial Sequence
  N-term extended E200 +GS linker+IgG hinge
DFPGHNYTTRNILPGLNITSGSEPKSSDKTHT

SEQ ID NO: 52 - protein, 35 aa, Artificial Sequence
  N-term extended E200 +G4S linker+IgG hinge
DFPGHNYTTRNILPGLNITSGGGGSEPKSSDKTHT

SEQ ID NO: 53 - protein, 40 aa, Artificial Sequence
  N and C term extended E200+IgG hinge
DFPGHNYTTRNILPGLNITSTFHKTSGSGKEPKSSDKTHT

SEQ ID NO: 54 - protein, 42 aa, Artificial Sequence
  N and C term extended E200+GS linker+IgG hinge
DFPGHNYTTRNILPGLNITSTFHKTSGSGKGSEPKSSDKTHT

SEQ ID NO: 55 - protein, 45 aa, Artificial Sequence
  N and C term extended E200+ G4S linker+ IgG hinge
DFPGHNYTTRNILPGLNITSTFHKTSGSGKGGGGSEPKSSDKTHT

SEQ ID NO: 56 - protein, 32 aa, Artificial Sequence
  N-term extended E200+IgG hinge +GS linker
DFPGHNYTTRNILPGLNITSEPKSSDKTHTGS

SEQ ID NO: 57 - protein, 34 aa, Artificial Sequence
  N-term extended E200+GS linker+IgG hinge +GS linker
DFPGHNYTTRNILPGLNITSGSEPKSSDKTHTGS

SEQ ID NO: 58 - protein, 37 aa, Artificial Sequence
  N-term extended E200+G4S linker+IgG hinge +GS linker
DFPGHNYTTRNILPGLNITSGGGGSEPKSSDKTHTGS

SEQ ID NO: 59 - protein, 42 aa, Artificial Sequence
  N-term and C term extended E200 + IgG hinge+GS linker
DFPGHNYTTRNILPGLNITSTFHKTSGSGKEPKSSDKTHTGS

SEQ ID NO: 60 - protein, 44 aa, Artificial Sequence
  N-term and C term extended E200 +GS linker+ IgG hinge+GS linker
DFPGHNYTTRNILPGLNITSTFHKTSGSGKGSEPKSSDKTHTGS

SEQ ID NO: 61 - protein, 47 aa, Artificial Sequence
  N-term and C term extended E200 + G4S linker+IgG hinge+GS linker
DFPGHNYTTRNILPGLNITSTFHKTSGSGKGGGGSEPKSSDKTHTGS

SEQ ID NO: 62 - protein, 35 aa, Artificial Sequence
  N-term extended E200+IgG hinge +G4S linker
DFPGHNYTTRNILPGLNITSEPKSSDKTHTGGGGS

SEQ ID NO: 63 - protein, 37 aa, Artificial Sequence
  N-term extended E200+GS linker+IgG hinge +G4S linker
DFPGHNYTTRNILPGLNITSGSEPKSSDKTHTGGGGS

SEQ ID NO: 64 - protein, 40 aa, Artificial Sequence
  N-term extended E200+G4S linker+IgG hinge +G4S linker
DFPGHNYTTRNILPGLNITSGGGGSEPKSSDKTHTGGGGS

SEQ ID NO: 65 - protein, 45 aa, Artificial Sequence
  N-term and C term extended E200 + IgG hinge+G4S linker
DFPGHNYTTRNILPGLNITSTFHKTSGSGKEPKSSDKTHTGGGGS

SEQ ID NO: 66 - protein, 47 aa, Artificial Sequence
  N-term and C term extended E200 +GS linker+ IgG hinge+G4S linker
DFPGHNYTTRNILPGLNITSTFHKTSGSGKGSEPKSSDKTHTGGGGS

SEQ ID NO: 67 - protein, 50 aa, Artificial Sequence
  N-term and C term extended E200 + G4S linker+IgG hinge+G4S linker
DFPGHNYTTRNILPGLNITSTFHKTSGSGKGGGGSEPKSSDKTHTGGGGS

SEQ ID NO: 68 - protein, 32 aa, Artificial Sequence
  “Native peptide 1”
GHNYTTRNILPGLNITSTFHKTSGSGKGGGGS

SEQ ID NO: 69 - protein, 32 aa, Artificial Sequence
  “Native peptide 3”
GGGGSGHNYTTRNILPGLNITSTFHKTSGSGK

SEQ ID NO: 70 - protein, 35 aa, Artificial Sequence
  “Native peptide 4”
GGGGSDFPGHNYTTRNILPGLNITSTFHKTSGSGK

SEQ ID NO: 71 - protein, 5 aa, Artificial Sequence
  Linker motif 1
GSGGS

SEQ ID NO: 72 - protein, 5 aa, Artificial Sequence
  Linker motif 2
GGGGS

SEQ ID NO: 73 - protein, 4 aa, Artificial Sequence
  Linker motif 3
GGGS

SEQ ID NO: 74 - protein, 6 aa, Artificial Sequence
  (GS)3 linker
GSGSGS

SEQ ID NO: 75 - protein, 15 aa, Artificial Sequence
  (G4S)3 linker
GGGGSGGGGSGGGGS

SEQ ID NO: 76 - protein, 13 aa, Artificial Sequence
  TP5 linker
GTPTPTPTPTGEF

SEQ ID NO: 77 - protein, 17 aa, Artificial Sequence
  short and/or alpha-helical rigid linker 1
AEAAAKEAAAKEAAAKA

SEQ ID NO: 78 - protein, 5 aa, Artificial Sequence
  short and/or alpha-helical rigid linker 2
PAPAP

(19) United States
(12) Patent Application Publication
(10) Pub. No.: US 2025/0282844 A1
(43) Pub. Date: Sep. 11, 2025

(54) METHODS FOR CONTROLLING IMMUNE CELL ACTIVITY

(71) Applicant: BIOSCEPTRE (AUST) PTY LTD, North Ryde (AU)

(72) Inventors: Patrick Schlegel; Julian Alexander Barden; Ziduo Li

(21) Appl. No.: 18/285,621

(22) PCT Filed: Apr. 8, 2022
(86) PCT No.: PCT/AU22/50311

§ 371 Date: Oct. 4, 2023

(30) Foreign Application Priority Data
Apr. 8, 2021 (AU) 2021901030

(51) Int. Cl. C07K 14/705; A61K 38/00

(52) U.S. Cl. CPC C07K 14/705; A61K 38/00; C07K 2319/30

(57) ABSTRACT

The present invention relates to methods for inhibiting the activity of a cellular immunotherapeutic in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
wherein the epitope on the molecule competes with an epitope on the target antigen, for binding to the cellular immunotherapeutic and the molecule thereby disrupts the interaction between the cellular immunotherapeutic and the target antigen;
thereby inhibiting the activity of the cellular immunotherapeutic in the subject.

### RELATED APPLICATION

This application is a national stage filing under 35 U.S.C. § 371 of PCT/AU2022/050311, filed Apr. 8, 2022, which claims priority from Australian provisional application AU 2021901030, filed Apr. 8, 2021, the contents of each of which are hereby incorporated by reference in their entirety.

### FIELD OF THE INVENTION

The present invention relates to improved methods of treatment that comprise the administration of immune cells.

### SEQUENCE LISTING

The instant application contains a Sequence Listing which has been submitted electronically in ASCII format and is hereby incorporated by reference in its entirety. Said ASCII copy, created on Apr. 30, 2024, is named 53282063AZD_ST25_amended.txt and is 34,839 bytes in size.

### BACKGROUND OF THE INVENTION

Adoptive immunotherapy, which involves the transfer of antigen-specific T-cells generated ex vivo, is a promising strategy to treat cancer. The cells may be autologous, allogeneic or derived from an HLA-matched (or partially matched) third party. The T-cells used for adoptive immunotherapy can be generated either by expansion of antigen-specific T cells or redirection of T-cells through genetic engineering.

Novel specificities in T-cells have been successfully generated through the genetic transfer of transgenic T cell receptors or chimeric antigen receptors (CARs). CARs are synthetic receptors consisting of a targeting moiety that is associated with one or more signalling domains in a single fusion molecule. CARs have successfully allowed T-cells to be redirected towards antigens expressed at the surface of tumour cells from various malignancies including lymphomas and solid tumours.

Various generations of CAR architecture have been developed, each with the aim of enhancing the activation signal, proliferation, production of cytokines and effector function of CAR-modified T cell in preclinical trials. For example, second-generation CARs were developed to incorporate the intracellular domains of one or more costimulatory molecules such as CD28, OX40, and 4-1BB within the endodomain, and these improved antigen-specific T-cell activation and expansion. Third-generation CARs include a combination of costimulatory endodomains. Fourth-generation CARs include an activation-dependent cytokine secretion in addition to costimulatory endodomains. Fifth-generation CAR-T cells comprise deletions of the human leukocyte antigen (HLA) and TCR genes of T cells obtained from healthy donors in order to avoid host immune rejection or graft-vs-host disease (GvHD) against the transplanted CAR T cells.

Despite their promise for the treatment of cancer, various adverse events have been reported for CAR-modified T cells. In one example, a patient died 5 days after cyclophosphamide chemotherapy followed by infusion of CAR-modified T cells recognising the antigen ERBB2 (HER-2/neu). The toxicity lead to a clinically significant release of pro-inflammatory cytokines, pulmonary toxicity, multi-organ failure and eventual death of the patient. This and other adverse events highlight the need for caution when employing CAR-modified T cells, as unlike antibodies against tumour-associated antigens, these cells are not cleared from the body within a short amount of time.

Several studies report diverse systems that aim to improve the efficacy and safety of cellular immunotherapy. In one example, inhibitory chimeric antigen receptors (iCARs) were designed to halt/inhibit T cell function upon encountering off-target cells. The iCAR is made up of an antigen-specific single-chain variable fragment (scFv) fused to a T cell inhibitory signalling domain. Cells expressing a tumour-associated antigen but not a normal-tissue antigen induce T cell activation, cytotoxicity and cytokine signalling to kill the on-target cells. In order to function, the iCAR technology relies on a preliminary selection of 2 antigens: one tumour associated antigen and one normal-tissue antigen.

Another system includes the use of an anti-CD20 CAR combined with an inducible caspase 9 (iC9) suicide switch. The latter gene is made functional in the presence of the prodrug AP1903 (tacrolimus) by binding to the mutated FK506-binding protein (FKBP1). Viral transduction transfers DNA from a vector into the target cell and the vector-derived DNA directs expression of chemical induction dimerisation (CID) and accessory proteins. In the presence of the AP1903 drug, there will be a dimerisation of the CID proteins, thus turning on the signal cascade. In the event of a serious or life-threatening toxicity caused by the administered T cells, AP1903 will be infused to trigger rapid destruction and elimination of the CaspaCID™-enabled cells. A similar apoptosis-inducing system based on a multimerising agent is described in WO 2014/152177.

So-called “adaptor CAR” platforms have also been developed, comprising a modular design and in which the T cells are typically in an “off” state until an adaptor molecule is provided that directs the T cell to the tumour-associated antigen, and thereby facilitates activation of the CAR-T cell.

The approaches of the prior art typically involve complex inhibitory or “off-switch” mechanisms for controlling immune cell activity or alternatively require use of complex adaptor systems for switching CAR T cells on. Moreover, many of the approaches described in the prior art for “switching-off” CAR-T cell activity result in killing of the cells and thus do not provide for sufficient flexibility in the use of CAR-T cell immunotherapies.

There is a need for improved approaches for controlling the activity of immune cell-based therapies, particularly CAR-T cell-based therapies.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention is based on the recognition by the inventors that it is possible to fine-tune the activity of immune cells that recognise and bind to a particular target antigen on a cancer cell.

In a first aspect, the present invention provides various methods for dampening or temporarily “switching-off” or inhibiting the activity of a cellular immunotherapeutic, such as but not limited to a CAR T therapy.

In one embodiment of the first aspect of the invention, there is provided a method for inhibiting the activity of a cellular immunotherapeutic in a subject who has received or is receiving a therapy with a cellular immunotherapeutic, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
   wherein the epitope on the molecule competes with an epitope on the target antigen, for binding to the cellular immunotherapeutic and the molecule thereby disrupts the interaction between the cellular immunotherapeutic and the target antigen;
   thereby inhibiting the activity of the cellular immunotherapeutic in the subject.

Preferably, the inhibition is reversible, such that once the molecule has been cleared from the circulation of the subject, the activity of the cellular immunotherapeutic is reinstated. Preferably, the method does not reduce the viability of the cells of the cellular immunotherapeutic.

The skilled person will be familiar with various cytotoxic events that may be triggered or caused by cellular immunotherapies. Such cytotoxic events may include any aberrant release of cytokines or inflammatory response following administration of a cellular immunotherapy. The aberrant release of cytokines may be referred to by various terms including hypercytokinemia or “cytokine storm”. “Cytokine release syndrome” (CRS) is a form of systemic inflammatory response syndrome (SIRS) and occurs when large numbers of white blood cells are activated and release inflammatory cytokines, which in turn activate yet more white blood cells. The term “cytokine storm” is often loosely used interchangeably with cytokine release syndrome (CRS) but is more precisely a differentiable syndrome that may represent a severe episode of cytokine release syndrome or a component of another disease entity, such as macrophage activation syndrome. When occurring as a result of a therapy, CRS symptoms may be delayed until days or weeks after treatment. Immediate-onset (fulminant) CRS appears to be a cytokine storm.

Accordingly, in a further embodiment of the first aspect of the invention, there is provided a method for minimising or reducing the risk of an aberrant inflammatory response in a subject who has received or is receiving a therapy with a cellular immunotherapeutic for binding to a target antigen on a cell, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
   wherein the epitope on the molecule competes with an epitope on the target antigen for binding to the cellular immunotherapeutic and the molecule thereby disrupts the interaction between the cellular immunotherapeutic and the target antigen;
   thereby minimising or reducing the risk of an aberrant inflammatory response in the subject. Preferably, the method does not reduce the viability of the cells of the cellular immunotherapeutic.

Further, there is provided a method for treating an aberrant inflammatory response in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
   wherein the epitope on the molecule competes with an epitope on the target antigen for binding to the cellular immunotherapeutic and the molecule thereby disrupts the interaction between the cellular immunotherapeutic and the target antigen;
   thereby treating an aberrant inflammatory response in the subject. Preferably, the method does not reduce the viability of the cells of the cellular immunotherapeutic.

The aberrant inflammatory response may comprise a cytokine-associated toxicity. The inflammatory response may comprise a systemic inflammatory response, and may be classified as Systemic Inflammatory Response Syndrome (SIRS). The SIRS may be classified as “cytokine release syndrome” (CRS). The aberrant inflammatory response may be hypercytokinemia or “cytokine storm”.

The aberrant inflammatory response may comprise one or more of the symptoms listed in Table 1 herein.

It will be appreciated that in further embodiments, a periodic and reversible “switching-off” of a cellular therapeutic may be desirable in order to increase or maximise the persistence of the cellular therapeutic in a subject (in other words, a period of inactivity for the cellular therapeutic), prevents exhaustion of the immune cells and enables the cells to recover potency.

Accordingly, the invention provides a method for promoting or increasing the persistence of a cellular immunotherapeutic in a subject, wherein the cellular immunotherapeutic is for binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
   wherein the epitope on the molecule competes for binding to the cellular immunotherapeutic and the molecule thereby disrupts the interaction between the cellular immunotherapeutic and the target antigen;
   thereby promoting or increasing the persistence of the cellular immunotherapeutic in the subject.

It will be understood that by administering the molecule that competes with the target antigen for binding to the cellular immunotherapeutic, the method provides for a period of time in which the cellular immunotherapeutic is no longer active, or has reduced activity, which thereby promotes the persistence of the cellular immunotherapeutic, as further explained herein.

Further, there is provided a use of a molecule comprising or consisting of an epitope of a target antigen, in the manufacture of a medicament for:
a) inhibiting the activity of a cellular immunotherapeutic in a subject who has received or is receiving a therapy with a cellular immunotherapeutic for binding to the target antigen;
b) minimising or reducing the risk of an aberrant inflammatory response in a subject who has received or is receiving a therapy with a cellular immunotherapeutic for binding to the target antigen;
c) treating an aberrant inflammatory response in a subject who has received or is receiving a therapy with a cellular immunotherapeutic for binding to the target antigen;
d) minimising or reducing the risk of tumour lysis syndrome in a subject who has received or is receiving a therapy with a cellular immunotherapeutic for binding to the target antigen;
e) treating tumour lysis syndrome in a subject who has received or is receiving a therapy with a cellular immunotherapeutic for binding to the target antigen; or
f) increasing the persistence of a cellular immunotherapeutic in a subject, wherein the cellular immunotherapeutic is for binding to the target antigen;
   wherein the molecule competes with the target antigen for binding to the cellular immunotherapeutic and the molecule thereby disrupts the interaction between the cellular immunotherapeutic and the target antigen;
   wherein preferably, the molecule does not target the cellular immunotherapeutic for cell-mediated killing.

Further still there is provided a composition comprising a molecule that comprises or consists of an epitope of a target antigen, for use in:
a) inhibiting the activity of a cellular immunotherapeutic in a subject who has received or is receiving a therapy with a cellular immunotherapeutic for binding to the target antigen;
b) minimising or reducing the risk of an aberrant inflammatory response in a subject who has received or is receiving a therapy with a cellular immunotherapeutic for binding to the target antigen;
c) treating an aberrant inflammatory response in a subject who has received or is receiving a therapy with a cellular immunotherapeutic for binding to the target antigen;
d) minimising or reducing the risk of tumour lysis syndrome in a subject who has received or is receiving a therapy with a cellular immunotherapeutic for binding to the target antigen;
e) treating tumour lysis syndrome in a subject who has received or is receiving a therapy with a cellular immunotherapeutic for binding to the target antigen on a cell; or
f) increasing the persistence of a cellular immunotherapeutic in a subject, wherein the cellular immunotherapeutic is for binding to the target antigen;
   wherein the molecule competes with the target antigen for binding to the cellular immunotherapeutic and the molecule thereby disrupts the interaction between the cellular immunotherapeutic and the target antigen;
   wherein preferably, the molecule does not target the cellular immunotherapeutic for cell-mediated killing.

In further embodiments of the first aspect of the invention, there is provided a kit for use in a method described herein, the kit comprising:
a cellular immunotherapeutic for binding to a target antigen; and
a molecule for binding to the cellular immunotherapeutic, the molecule comprising or consisting of an epitope of the target antigen;
   wherein the epitope on the molecule competes with the target antigen for binding to the cellular immunotherapeutic and the molecule thereby disrupts the interaction between the cellular immunotherapeutic and the target antigen.

Optionally, the kit comprises written instructions for use in a method of the first aspect of the invention.

In any embodiment of the first aspect, the molecule reduces the ability of the cellular immunotherapeutic to bind to a target antigen on a cell, particularly wherein the target antigen is expressed or present on the surface of a cancer cell. Preferably the molecule blocks the interaction of the cellular immunotherapeutic with the target antigen present on the cell. In other words, the cellular immunotherapeutic comprises a moiety for binding to the target antigen on a cancer cell and the molecule comprises a similar or the same epitope as the target antigen, such that the molecule competes with the target antigen on the cell for binding to the cellular immunotherapeutic. Accordingly, the methods of the invention comprise molecules for displacing or preventing or reducing the binding of a cellular immunotherapeutic to its target antigen on cancer cells.

Preferably, the cell that comprises the target antigen is a cancer cell and the cellular immunotherapeutic is for use in the treatment of cancer. Examples of such cellular immunotherapeutics for binding to target antigens on cancer cells include CAR T cells, indirect, or ligand-based CAR-T cells, cells with modified TCRs and other cellular therapeutics as further defined herein.

It will be appreciated that various formats of molecule may be utilised in accordance with the present invention, provided that the molecule comprises an epitope of the target antigen that competes for binding by the cellular immunotherapeutic.

In any embodiment, the molecule for binding to the cellular immunotherapeutic, comprises or consists of a polypeptide. The polypeptide may be in the form of a fusion or chimeric protein or any other polypeptide molecule.

In certain embodiments, where the molecule is a polypeptide, the methods may comprise providing a nucleic acid encoding the polypeptide, to the subject.

It will be appreciated that a wide variety of other architectures could be employed for the molecule that comprises the epitope, for example, the molecule may be in the form of a polypeptide comprising the epitope, conjugated or fused to any suitable carrier moiety. In certain embodiments, the carrier moiety may be selected from: a carbohydrate, a lipid, a liposome, a peptide, and an aptamer, as further defined herein.

In some embodiments, the epitope may be provided in the context of a liposome (e.g., a pegylated liposome) comprising the epitope on the surface of PEG couplings. This provides for a liposome that is coated in the epitope that is recognised and bound by the cellular immunotherapeutic.

In any embodiment of the first aspect, the molecule is in the form of a fusion protein comprising the epitope that is specific to the target antigen. The fusion protein may comprise the epitope, and a further sequence for facilitating improved solubility of the polypeptide.

The further sequence may comprise an Fc region of an antibody. However, it will be appreciated that in accordance with the first aspect of the invention, it is preferable that the Fc region of an antibody be modified such that it does not exhibit effector function or any detectable effector function. For example, the Fc region of the antibody may be modified so that the region does not bind the Fc γ Receptor (FcγR), although it may retain FcRn binding. The Fc region of the antibody may be modified so that the region is not bound by various cells that are responsible for mediating antibody-dependent cell killing (ADCC), such as NK cells (which express FcγRIII only) or monocytes (which express FcγRI, FcγRII and FcγRIII) or haematopoietic cells. Preferably the Fc region is modified such that it does not trigger complement-dependent cytotoxicity (CDC).

The further sequence may comprise serum albumin, transferrin, a carboxy-terminal peptide of chorionic gonadotropin (CG) β chain, a non-exact repeat peptide sequence, a polypeptide sequence composed of proline-alanine-serine polymer, an elastin-like peptide (ELP) repeat sequence), a homopolymer of glycine residues or a gelatin-like protein.

In a second aspect, the present invention provides various methods for irreversible, or permanent “switching-off” of the activity of a cellular immunotherapeutic, (such as but not limited to a CAR T therapy), for binding to target antigen, preferably a target antigen on a cancer cell.

In one embodiment of the second aspect of the invention, there is provided a method for inhibiting the activity of a cellular immunotherapeutic in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic; wherein the molecule comprises:
i) an epitope of the target antigen that competes for binding to the cellular immunotherapeutic, and the molecule thereby disrupts the interaction between the cellular immunotherapeutic and the target antigen;
ii) a compound for triggering cell death,
   wherein, upon binding of the molecule to the cellular therapeutic, the cellular immunotherapeutic undergoes cell death or is targeted for cell-killing;
   thereby inhibiting the activity of the cellular immunotherapeutic in the subject.

Preferably, the inhibition is irreversible, such that the method reduces the viability of the cells of the cellular immunotherapeutic, optionally by introducing a toxin to the cellular immunotherapeutic or by targeting the cells for killing, for example by NK cells and other cytotoxic cells.

The skilled person will be familiar with various cytotoxic events that may be triggered or caused by cellular immunotherapies. Such cytotoxic events may include any aberrant release of cytokines or inflammatory response following administration of a cellular immunotherapy. The aberrant release of cytokines may be referred to by various terms including hypercytokinemia or “cytokine storm”. “Cytokine release syndrome” (CRS) is a form of systemic inflammatory response syndrome (SIRS) and occurs when large numbers of white blood cells are activated and release inflammatory cytokines, which in turn activate yet more white blood cells. The term “cytokine storm” is often loosely used interchangeably with cytokine release syndrome (CRS) but is more precisely a differentiable syndrome that may represent a severe episode of cytokine release syndrome or a component of another disease entity, such as macrophage activation syndrome. When occurring as a result of a therapy, CRS symptoms may be delayed until days or weeks after treatment. Immediate-onset (fulminant) CRS appears to be a cytokine storm.

Accordingly, in a further embodiment of the second aspect of the invention, there is provided a method for minimising or reducing the risk of an aberrant inflammatory response in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic; wherein the molecule comprises:
i) an epitope of the target antigen that competes for binding to the cellular immunotherapeutic, and the molecule thereby disrupts the interaction between the cellular immunotherapeutic and the target antigen;
ii) a compound for triggering cell death,
   wherein, upon binding of the molecule to the cellular therapeutic, the cellular immunotherapeutic undergoes cell death or is targeted for cell-mediated killing;
   thereby minimising or reducing the risk of an aberrant inflammatory response in the subject.

Preferably, the method reduces the viability of the cells of the cellular immunotherapeutic, preferably targeting the cells for degradation/killing and thereby avoiding or reducing risk of further aberrant immune responses that may otherwise be triggered by the cellular immunotherapeutic.

Further, there is provided a method for treating an aberrant inflammatory response in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic; wherein the molecule comprises:
i) an epitope of the target antigen that competes for binding to the cellular immunotherapeutic, and the molecule thereby disrupts the interaction between the cellular immunotherapeutic and the target antigen;
ii) a compound for triggering cell death,
   wherein, upon binding of the molecule to the cellular therapeutic, the cellular immunotherapeutic undergoes cell death or is targeted for cell-mediated killing;
   thereby treating an aberrant inflammatory response in the subject.

Preferably, the method reduces the viability of the cells of the cellular immunotherapeutic, preferably targeting the cells for degradation/killing.

The aberrant inflammatory response may comprise a cytokine-associated toxicity. The inflammatory response may comprise a systemic inflammatory response, and may be classified as Systemic Inflammatory Response Syndrome (SIRS). The SIRS may be classified as “cytokine release syndrome” (CRS). The aberrant inflammatory response may be hypercytokinemia or “cytokine storm”.

The aberrant inflammatory response may comprise one or more of the symptoms listed in Table 2 herein.

Further, there is provided a use of a molecule comprising an epitope of a target antigen, in the manufacture of a medicament for:
a) inhibiting the activity of a cellular immunotherapeutic in a subject who has received or is receiving a therapy with a cellular immunotherapeutic for binding to the target antigen;
b) minimising or reducing the risk of an aberrant inflammatory response in a subject who has received or is receiving a therapy with a cellular immunotherapeutic for binding to the target antigen; or
c) treating an aberrant inflammatory response in a subject who has received or is receiving a therapy with a cellular immunotherapeutic for binding to the target antigen;
d) minimising or reducing the risk of tumour lysis syndrome in a subject who has received or is receiving a therapy with a cellular immunotherapeutic for binding to the target antigen; or
e) treating tumour lysis syndrome in a subject who has received or is receiving a therapy with a cellular immunotherapeutic for binding to the target antigen;
   wherein the molecule comprises:
i) an epitope of the target antigen that competes with the target antigen for binding to the cellular immunotherapeutic, and the molecule thereby disrupts the interaction between the cellular immunotherapeutic and the target antigen;
ii) a compound for triggering cell death,
   wherein, upon binding of the molecule and the cellular therapeutic, the cellular immunotherapeutic undergoes cell death or is targeted for cell-mediated killing.

Further still there is provided a composition comprising a molecule that comprises an epitope of a target antigen, for use in:
a) inhibiting the activity of a cellular immunotherapeutic in a subject who has received or is receiving a therapy with a cellular immunotherapeutic for binding to the target antigen;
b) minimising or reducing the risk of an aberrant inflammatory response in a subject who has received or is receiving a therapy with a cellular immunotherapeutic for binding to the target antigen;
c) treating an aberrant inflammatory response in a subject who has received or is receiving a therapy with a cellular immunotherapeutic for binding to the target antigen; or
d) minimising or reducing the risk of tumour lysis syndrome in a subject who has received or is receiving a therapy with a cellular immunotherapeutic for binding to the target antigen;
e) treating tumour lysis syndrome in a subject who has received or is receiving a therapy with a cellular immunotherapeutic for binding to the target antigen;
   wherein the molecule comprises:
i) an epitope of the target antigen that competes with the target antigen on the cell for binding to the cellular immunotherapeutic, and the molecule thereby disrupts the interaction between the cellular immunotherapeutic and the target antigen;
ii) a compound for triggering cell death,
   wherein, upon binding of the molecule to the cellular therapeutic, the cellular immunotherapeutic undergoes cell death or is targeted for cell-mediated killing.

In further embodiments of the second aspect of the invention, there is provided a kit for use in a method described herein, the kit comprising:
a cellular immunotherapeutic for binding to a target antigen; and
a molecule comprising an epitope of the target antigen
wherein the molecule comprises:
i) an epitope of the target antigen that competes with the target antigen on the cell for binding to the cellular immunotherapeutic, and the molecule thereby disrupts the interaction between the cellular immunotherapeutic and the target antigen;
ii) a compound for triggering cell death,
   wherein, upon binding of the molecule to the cellular therapeutic, the cellular immunotherapeutic undergoes cell death or is targeted for cell-mediated killing.

Optionally, the kit comprises written instructions for use in a method of the second aspect of the invention.

Preferably, the target antigen is a cancer cell and the cellular immunotherapeutic is for use in the treatment of cancer. Preferably the molecule blocks the interaction of the cellular immunotherapeutic with the target antigen present on the cell. In other words, the cellular immunotherapeutic comprises a moiety for binding to the target antigen on a cancer cell and the molecule comprises a similar or the same epitope as the target antigen, such that the molecule competes with the target antigen on the cell for binding to the cellular immunotherapeutic. Accordingly, the methods of the invention comprise molecules for displacing or preventing or reducing the binding of a cellular immunotherapeutic to its target antigen on cancer cells and targeting the cellular immunotherapeutic for cell death or cell-mediated killing.

In accordance with the second aspect of the invention, the compound for triggering cell death may be any toxin or chemotherapeutic that induces cytotoxicity. Accordingly, the molecule may be in the form of a protein conjugate comprising a first moiety comprising an epitope of the target antigen, and a second moiety in the form of a cytotoxic compound (e.g., a toxin or chemotherapeutic that induces apoptosis upon binding of the molecule). In further embodiments, the molecule may be in the form of an anti-idiotype antibody for binding to the cellular immunotherapeutic (i.e., preferably for binding to the region of the cellular immunotherapeutic that is responsible for binding to the target antigen). In such embodiments, the anti-idiotype antibody may comprise a toxin or chemotherapeutic conjugated thereto. Suitable toxins or chemotherapeutics are known to the skilled person (and may have found use in other contexts, such as antibody-drug conjugates).

In a preferred embodiment of the second aspect, the molecule is in the form of a fusion protein comprising the epitope that is specific to the target antigen and an amino acid sequence for triggering cell-mediated killing. The amino acid sequence for triggering cell-mediated killing may be any sequence that facilitates targeting of the cellular immunotherapeutic for cell-mediated killing, for example, by cytotoxic immune cells such as cytotoxic T-lymphocytes or NK cells, when the cellular immunotherapeutic is bound by the polypeptide.

The further sequence preferably comprise an Fc region of an antibody, and in such embodiments, the polypeptide may be referred to as an “Fc-fusion protein comprising an epitope that is specific to a dysfunctional P2X₇ receptor”. It will be appreciated that in accordance with the second aspect of the invention, it is preferable that the Fc region of an antibody exhibits effector function and binds the Fcγ Receptor (FCγR). The Fc region of the antibody preferably comprises sequences that are bound by various cells that are responsible for mediating antibody-dependent cell killing (ADCC), such as NK cells (which express FcγRIII only) or monocytes (which express FcγRI, FcγRII and FcγRIII) or haematopoietic cells. The Fc region of an antibody may also comprise a sequence that triggers complement-dependent cytotoxicity (CDC).

In accordance with the first or second aspects of the invention, the target antigen may be any antigen associated with a cancer cell and which can be used as a target for binding by a cellular immunotherapeutic.

Examples of target antigens for binding by a cellular immunotherapeutic include, but are not limited to: dysfunctional (nf)P2X₇ receptor, mesothelin, EGFR, GPC3, MUC1, HER2, GD2, CEA, EpCAM, LeY, PCSA, CD19, CD20, Clec9a, CD276, PD-L1 and PD-L2. Other examples of target antigens are further described herein.

In any embodiment of the first and second aspects, the cellular immunotherapeutic comprises an immune cell, or progenitor thereof, wherein the immune cell expresses a receptor comprising an antigen-recognition domain and a signalling domain, wherein the antigen-recognition domain binds to a target antigen expressed on cancer cell surface.

In alternative embodiments of the first and second aspects, the cellular immunotherapeutic comprises an immune cell, or progenitor thereof, wherein the immune cell expresses a receptor comprising an antigen-recognition domain and a signalling domain, wherein the antigen-recognition domain binds to a ligand which facilitates binding of the immune cell to a target antigen expressed on cancer cell surface.

The immune cell or progenitor thereof may be a T cell, an NK cell, or any other immune cell that expresses a receptor or binding domain for binding to a target antigen on a cancer cell.

Typically, the antigen-recognition domain of the receptor expressed by the cellular immunotherapeutic solely recognises a tumour-associated antigen as herein defined.

In some embodiments, the signalling domain includes a portion derived from an activation receptor. In some embodiments, the activation receptor is a member of the CD3 co-receptor complex or is an Fc receptor. In some embodiments, the portion derived from the CD3 co-receptor complex is CD3-ζ. In some embodiments, the portion derived from the Fc receptor is FcεRI or FcγRI.

In some embodiments, the signalling domain includes a portion derived from a co-stimulatory receptor. In some embodiments, the signalling domain includes a portion derived from an activation receptor and a portion derived from a co-stimulatory receptor. In some embodiments, the co-stimulatory receptor is selected from the group consisting of CD27, CD28, CD30, CD40, DAP10, OX40, 4-1BB (CD137) and ICOS.

In any embodiment, the receptor expressed by the immune cell may be a chimeric antigen receptor (CAR) that binds to a target antigen expressed on a cell surface and the cellular immunotherapeutic therefore comprises an immune cell expressing a CAR. Other receptors (such as modified TCRs or ligand-based CARs) are also contemplated within the scope of the present invention.

In any embodiment, the immune cell expressing the antigen receptor may be any immune cell as described herein, or a cell that is capable of differentiating into an immune cell (e.g., a progenitor of an immune cell). A cell that is capable of differentiating into an immune cell (e.g. T cell that will express the CAR) may be a stem cell, multi-lineage progenitor cell or induced pluripotent stem cell.

In any embodiment, the immune cell expressing a CAR may be any immune cell as described herein, or a cell that is capable of differentiating into an immune cell (e.g., a progenitor of an immune cell). A cell that is capable of differentiating into an immune cell (e.g. T cell that will express the CAR) may be a stem cell, multi-lineage progenitor cell or induced pluripotent stem cell. As such, in particularly preferred embodiments, the cellular immunotherapeutic comprises a CAR-T cell that binds a tumour associated antigen.

In certain embodiments, the cellular immunotherapeutic is for binding to dysfunctional P2X₇ receptors on cancer cells and the antigen-recognition domain of the receptor expressed by the cellular immunotherapeutic therefore binds to dysfunctional P2X₇ receptors.

In any embodiment, the antigen-recognition domain binds to an epitope associated with an adenosine triphosphate (ATP)-binding site of the dysfunctional P2X₇ receptor. In some embodiments, the dysfunctional P2X₇ receptor has a reduced capacity to bind ATP at the ATP-binding site compared with an ATP-binding capacity of a functional P2X₇ receptor (e.g., a receptor having wild-type sequence and having a conformation or fold of an ATP-binding receptor). In some embodiments the dysfunctional P2X₇ receptor cannot bind ATP at the ATP-binding site.

In any embodiment, the dysfunctional P2X₇ receptor has a conformational change that renders the receptor dysfunctional. In some embodiments, the conformational change is a change of an amino acid from the trans-conformation to the cis-conformation. In some embodiments, the amino acid that has changed from a trans-conformation to a cis-conformation is proline at amino acid position 210 of the dysfunctional P2X₇ receptor.

In any embodiment, the antigen-recognition domain binds to an epitope that includes the proline at amino acid position 210 of the dysfunctional P2X₇ receptor. In some embodiments, the antigen-recognition domain binds to an epitope that includes one or more amino acid residues spanning from glycine at amino acid position 200 to cysteine at amino acid position 216, inclusive, of the dysfunctional P2X₇ receptor.

The antigen-recognition domain of the receptor can be any suitable molecule that can interact with and specifically binds to a dysfunctional P2X₇ receptor. However, in some embodiments, the antigen-recognition domain includes amino acid sequence homology to the amino acid sequence of an antibody, or a fragment thereof, which binds to the dysfunctional P2X₇ receptor. In some embodiments, the antigen-recognition domain includes amino acid sequence homology to the amino acid sequence of a fragment-antigen binding (Fab) portion of an antibody that binds to a dysfunctional P2X₇ receptor. In some embodiments, the antibody is a humanised antibody.

In any embodiment, the antigen-recognition domain includes amino acid sequence homology to the amino acid sequence of a single-chain variable fragment (scFv) or a multivalent scFv that binds to a dysfunctional P2X₇ receptor. In some embodiments, the multivalent scFv is a divalent or trivalent scFv.

In any embodiment, the antigen-recognition domain includes amino acid sequence homology to a single-antibody domain (sdAb) that binds to a dysfunctional P2X₇ receptor.

In any embodiment, the antigen-recognition domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor. In preferred embodiments, the binding polypeptide comprises the amino acid sequence of the CDRs of the V_{H} and/or V_{L} chain of an antibody, or the amino acid sequence of the V_{H} and/or V_{L} chains of an antibody, or the amino acid sequence of an antibody or fragment thereof, wherein the antibody or fragment thereof comprises the amino acid sequences of any antibody described in PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. No. 8,440,186, 9,181,320, 9,944,701 or 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. No. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In accordance with the first and second aspects of the invention, the methods involve the use of a molecule or a polypeptide that competes for binding to the cellular immunotherapeutic, and displaces or blocks binding of the cellular immunotherapeutic to the target antigen, preferably wherein the target antigen is expressed or present on the surface of a cancer cell.

The molecule or polypeptide preferably comprises an epitope of the target antigen, so that the molecule or polypeptide is preferentially bound by the cellular immunotherapeutic, thereby liberating the cellular immunotherapeutic from its binding to the cancer cell.

It will be well within the purview of the skilled person to generate a molecule or polypeptide (or nucleic acid encoding the polypeptide as the case may be), comprising an epitope of the target antigen for binding by the cellular immunotherapeutic. For example, in the context of a cellular immunotherapeutic for binding to CD19, the molecule or polypeptide will comprise a similar epitope of the CD19 molecule that is recognised by the cellular immunotherapeutic, so that the cellular immunotherapeutic preferentially binds to the molecule or polypeptide rather than CD19 present on the surface of target cells.

The following statements relate to the specific example whereby the target antigen is dysfunctional P2X₇ receptor however it will be appreciated that the scope of the invention is not limited to these examples.

In any embodiment of the first or second aspects of the invention, the cellular immunotherapeutic is for binding dysfunctional P2X₇ receptor on a cancer cell. Accordingly, in such embodiments, the molecule or polypeptide comprises an epitope of dysfunctional P2X₇ receptor that competes for binding to the cellular therapeutic.

In any embodiment of the first or second aspects, the epitope of the target antigen on the molecule comprises an amino acid sequence that is substantially the same, or homologous to the epitope on the dysfunctional P2X₇ receptor bound by the cellular immunotherapeutic. In other words, even though the amino acid sequence of the two epitopes may differ, there is sufficient homology for the cellular immunotherapeutic to bind to both the polypeptide and the dysfunctional P2X₇ receptor on a cell. In any embodiment, the epitope on the polypeptide comprises or consists of the amino acid sequence of the epitope on the dysfunctional P2X₇ receptor to which the cellular immunotherapeutic binds.

In any embodiment of the first or second aspects, the epitope of a dysfunctional P2X₇ receptor, comprises or consists of an epitope that is only found on dysfunctional P2X₇ receptor but is not found on a functional form of the P2X₇ receptor. In other words, preferably, the polypeptide comprises or consists of an epitope that is specific to a dysfunctional P2X₇ receptor.

In further embodiments of the first or second aspects, the polypeptide comprises an epitope corresponding to the E200, E300 or composite E200/E300 epitopes as herein defined. It will be within the purview of the skilled person to obtain various polypeptides for use in accordance with the invention, and particularly, in the context of “blocking” or reducing the binding efficacy of an anti-nfP2X₇ CAR. For example the skilled person will appreciate that it is possible to include additional amino acids N- or C-terminal to the region of the polypeptide comprising the epitope bound by the CAR. In a non-limiting example, and in the context of E200, which is typically defined as having an amino acid sequence substantially as defined in SEQ ID NO: 2 (and having a minimum sequence as defined in SEQ ID NO: 11), additional amino acids derived from the native sequence of P2X₇ receptor can be included in the polypeptide, for example, the residues “DFP” N-terminal to the epitope in the P2X₇ receptor sequence and/or residues “TFHKT” C-terminal to the epitope in the P2X₇ receptor sequence. In any embodiment, the polypeptide may comprise at least 1, at least 2, at least 3, at least 4, at least 5 or at least 6 amino acids derived from the P2X₇ receptor sequence, in addition to the sequence of the E200 or E300 or composite epitopes.

In a preferred embodiment, the sequence of the E200 epitope is further modified to substitute the cysteine residue (residue 17 in SEQ ID NO: 2) to a serine residue. The skilled person will appreciate that this can be done to reduce likelihood of any disulphide bonding between the polypeptide and another molecule.

It will also be within the purview of the skilled person to include additional amino acid residues to the E200, E300 or composite epitopes (or extended epitopes as discussed in the paragraph above), such as, for example, by the addition of at least 1, at least 2, at least 3, at least 4, at least 5, at least 6, at least 7, at least 8, at least 9 or at least 10 additional amino acid residues to the N- and C-terminal regions of peptides consisting of the amino acid sequence of the relevant epitope. Typically such additional amino acids can be derived from linker sequences (such as peptides comprising glycine and serine residues); or be derived from the hinge region of an immunoglobulin. Typically no more than 30, no more than 25, or no more than 20 amino acid residues are added to the N- and/or C-terminal residues of the E200, E300 or composite epitopes as defined herein.

It will be appreciated that in accordance with the first aspect of the invention, the molecule may be in the form of a polypeptide that consists of an epitope of a dysfunctional P2X₇ receptor.

Examples of peptides and polypeptides comprising epitopes of a dysfunctional P2X₇ receptor and suitable for use in accordance with the present invention, are provided in Table 1 (and are defined according to any of SEQ ID NOs: 2 to 70).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### DESCRIPTION OF THE DRAWINGS

FIG. 1: A. Percentage change in viability of MOLM-13 cells following co-culture in a direct CAR system with anti-nfP2X₇ CAR T cells+/−peptides comprising an epitope that is recognised by the anti-nfP2X₇ CAR. The presence of the different peptide variants reduced the killing of MOLM-13 cells by the CAR T cells. B. Percentage change in viability of MOLM-13 cells following co-culture in an indirect CAR T cell system (anti-nfP2X₇ CAR T cell+polypeptide comprising epitope for anti-nfP2X₇ CAR and an anti-CD33 binding domain)+/−peptides comprising an epitope that is recognised by the anti-nfP2X₇ CAR. The presence of the different peptide variants reduces the killing of MOLM-13 cells by the CAR T cells.

FIG. 2: Change in viability of MOLM-13 cells following co-culture with anti-nfP2X₇ CAR T cells+/−peptides comprising an epitope that is recognised by the anti-nfP2X₇ CAR.

FIG. 3: Killing of JeKo-1 cells by T cells and by T cells expressing an anti-nfP2X₇ CAR, in the presence or absence of a peptide comprising an epitope that is recognised by the CAR. Only T cells expressing nfP2X₇ CAR completely prevented proliferation of the JeKo-1 cells over the course of a 5 day period. Addition of the peptide (comprising the sequence of an epitope recognised by the nfP2X₇ CAR) reduced killing of the JeKo-1 cells by the CAR T cells.

FIG. 4: Reduction of the killing of MOLM-13 cells by nfP2X₇ CAR T cells is dose-dependent and can be controlled by varying the concentration of the peptide comprising the epitope recognised by the nfP2X₇ CAR.

### SEQUENCE INFORMATION

TABLE 1
Sequence information of exemplary dysfunctional P2X7 receptor epitope
sequences and peptides/polypeptides comprising same

| Description | Sequence | SEQ ID NO: |
|---|---|---|
| Human P2X₇ | MPACCSCSDVFQYETNKVTRIQSMNYGTIKWFFHVIIFSYVCFALV | 1 |
| receptor | SDKLYQRKEPVISSVHTKVKGIAEVKEEIVENGVKKLVHSVFDTAD | |
| E200 underlined | YTFPLQGNSFFVMTNFLKTEGQEQRLCPEYPTRRTLCSSDRGCK | |
| | KGWMDPQSKGIQTGRCVVYEGNQKTCEVSAWCPIEAVEEAPRP | |
| | ALLNSAENFTVLIKNNIDFPGHNYTTRNILPGLNITCTFHKTQNPQC | |
| | PIFRLGDIFRETGDNFSDVAIQGGIMGIEIYWDCNLDRWFHHCRPK | |
| | YSFRRLDDKTTNVSLYPGYNFRYAKYYKENNVEKRTLIKVFGIRFD | |
| | ILVFGTGGKFDIIQLVVYIGSTLSYFGLAAVFIDFLIDTYSSNCCRSHI | |
| | YPWCKCCQPCVVNEYYYRKKCESIVEPKPTLKYVSFVDESHIRM | |
| | VNQQLLGRSLQDVKGQEVPRPAMDFTDLSRLPLALHDTPPIPGQ | |
| | PEEIQLLRKEATPRSRDSPVWCQCGSCLPSQLPESHRCLEELCC | |
| | RKKPGACITTSELFRKLVLSRHVLQFLLLYQEPLLALDVDSTNSRL | |
| | RHCAYRCYATWRFGSQDMADFAILPSCCRWRIRKEFPKSEGQY | |
| | SGFKSPY | |
| Exemplary E200 | GHNYTTRNILPGLNITC | 2 |
| epitope | | |
| Variant E200 | GHNYTTRNILPGLNIT | 3 |
| epitope peptide | | |
| (E200′) | | |
| E200 epitope Cys | GHNYTTRNILPGLNITS | 4 |
| to Ser | | |
| modification | | |
| Extended E200 | GHNYTTRNILPGLNITSTFHK | 5 |
| Cys to Ser | | |
| modification | | |
| Extended E200′ | GHNYTTRNILPGLNITSTFHKT | 6 |
| Cys to Ser | | |
| modification | | |
| (22 aa) | | |
| Extended E200″ | GHNYTTRNILPGLNITSTFHKTC | 7 |
| Cys to Ser | | |
| modification | | |
| Pep16 | DFPGHNYTTRNILPGC | 8 |
| Pep17 | GHNYTTRNILPGLNITSTFHKTS | 9 |
| Extended Pep17 + | GHNYTTRNILPGLNITSTFHKTSGSGK | 10 |
| linker region (C | | |
| term extended | | |
| E200 underlined) | | |
| Minimum | NYTTRNILPGL | 11 |
| sequence E200 | | |
| peptide (target | | |
| epitope) | | |
| Exemplary E300 | KYYKENNVEKRTLIK | 12 |
| epitope | | |
| Variant E300 | KYYKENNVEKRTLIKVF | 13 |
| epitope peptide | | |
| (E30′) | | |
| Exemplary | GHNYTTRNILPGAGAKYYKENNVEK | 14 |
| E200/E300 or | | |
| composite epitope | | |
| E200 (underlined) + | GHNYTTRNILPGLNITSGGGGS | 15 |
| G4S linker | | |
| E200 (underlined) + | GHNYTTRNILPGLNITSGGGGSGGGGS | 16 |
| 2xG4S linker | | |
| E200 (underlined) + | GHNYTTRNILPGLNITSGGGGSGGGGSGGGGS | 17 |
| 3xG4S linker | | |
| E200_extended | GHNYTTRNILPGLNITSTFHKTGS | 18 |
| peptide | | |
| (underlined) 17v3 | | |
| (24 aa) | | |
| E200_extended | GHNYTTRNILPGLNITSTFHGS | 19 |
| peptide | | |
| (underlined) 17v4 | | |
| (22 aa) | | |
| E200_extended | GHNYTTRNILPGLNITSGS | 20 |
| peptide | | |
| (underlined) 17v5 | | |
| (19 aa) | | |
| N term | DFPGHNYTTRNILPGLNITSGS | 21 |
| E200_extended | | |
| peptide | | |
| (underlined) 17v6 | | |
| (22 aa) | | |
| (E200 epitope | | |
| extended into N | | |
| terminal region) | | |
| E200_extended | DFPGHNYTTRNILPGLNITSGGGGS | 22 |
| peptide | | |
| (underlined) 17v7 | | |
| (25 aa) + linker | | |
| E200_extended | DFPGHNYTTRNILPGLNITSGGGGGGGGS | 23 |
| peptide | | |
| (underlined) 17v8 | | |
| (30 aa) + linker | | |
| E200_extended | DFPGHNYTTRNILPGLNITSGGGGSGGGGSGGGGS | 24 |
| peptide | | |
| (underlined) 17v9 | | |
| (35 aa) + linker | | |
| N and C term | DFPGHNYTTRNILPGLNITSTFHKTSGSGK | 25 |
| E200_extended | | |
| peptide | | |
| (underlined) | | |
| 17v10 (30 aa) | | |
| E200_extended | DFPGHNYTTRNILPGLNITSTFHKTSGSGKGS | 26 |
| peptide | | |
| (underlined) | | |
| 17v11 (32 aa) + | | |
| linker | | |
| E200_extended | DFPGHNYTTRNILPGLNITSTFHKTSGSGKGGGGS | 27 |
| peptide 17v12 (35 | | |
| aa) + linker | | |
| “Native peptide 2” | | |
| Extended E200 | | |
| underlined | | |
| E200_extended | DFPGHNYTTRNILPGLNITSTFHGGGGS | 28 |
| peptide 17v13 (25 | | |
| aa) + linker | | |
| E200_extended | GHNYTTRNILPGLNITSTFHGGGGS | 29 |
| peptide 17v14 (22 | | |
| aa) + linker | | |
| E200_extended | DFPGHNYTTRNILPGLNITSTFHKTGGGGS | 30 |
| peptide 17v15 (30 | | |
| aa) + linker | | |
| E200_extended | GHNYTTRNILPGLNITSTFHKTGGGGS | 31 |
| peptide 17v16 (27 | | |
| aa) + | | |
| E200 + IgG hinge | GHNYTTRNILPGLNITSEPKSSDKTHT | 32 |
| (E200 underlined) | | |
| E200 + GS | GHNYTTRNILPGLNITSGSEPKSSDKTHT | 33 |
| linker_IgG hinge | | |
| E200 + G4S | GHNYTTRNILPGLNITSGGGGSEPKSSDKTHT | 34 |
| linker + IgG hinge | | |
| Extended E200 + | GHNYTTRNILPGLNITSTFHKTSGSGKEPKSSDKTHT | 35 |
| _IgG hinge | | |
| Extended E200 + | GHNYTTRNILPGLNITSTFHKTSGSGKGSEPKSSDKTHT | 36 |
| GS linker + _IgG | | |
| hinge | | |
| Extended E200 + | GHNYTTRNILPGLNITSTFHKTSGSGKGGGGSEPKSSDKTHT | 37 |
| G4S linker + IgG | | |
| hinge | | |
| E200 + IgG | GHNYTTRNILPGLNITSEPKSSDKTHTGS | 38 |
| hinge + GS linker | | |
| E200 + GS | GHNYTTRNILPGLNITSGSEPKSSDKTHTGS | 39 |
| linker + IgG | | |
| hinge + GS linker | | |
| E200 + G4Slinker + | GHNYTTRNILPGLNITSGGGGSEPKSSDKTHTGS | 40 |
| IgG hinge + GS | | |
| linker | | |
| Extended | GHNYTTRNILPGLNITSTFHKTSGSGKEPKSSDKTHTGS | 41 |
| E200 + _IgG | | |
| hinge + GSlinker | | |
| Extended | GHNYTTRNILPGLNITSTFHKTSGSGKGSEPKSSDKTHTGS | 42 |
| E200 + GS | | |
| linker + _IgG | | |
| hinge + GSlinker | | |
| Extended | GHNYTTRNILPGLNITSTFHKTSGSGKGGGGSEPKSSDKTHTGS | 43 |
| E200 + G4S | | |
| linker + _IgG | | |
| hinge + GSlinker | | |
| E200 + IgG | GHNYTTRNILPGLNITSEPKSSDKTHTGGGGS | 44 |
| hinge + G4S linker | | |
| E200 + GS | GHNYTTRNILPGLNITSGSEPKSSDKTHTGGGGS | 45 |
| linker + IgG | | |
| hinge + G4S linker | | |
| E200 + G4S | GHNYTTRNILPGLNITSGGGGSEPKSSDKTHTGGGGS | 46 |
| linker + IgG | | |
| hinge + G4S linker | | |
| Extended | GHNYTTRNILPGLNITSTFHKTSGSGKEPKSSDKTHTGGGGS | 47 |
| E200 + IgG | | |
| hinge + G4S linker | | |
| Extended | GHNYTTRNILPGLNITSTFHKTSGSGKGSEPKSSDKTHTGGGGS | 48 |
| E200 + GS | | |
| linker + IgG | | |
| hinge + G4S linker | | |
| Extended | GHNYTTRNILPGLNITSTFHKTSGSGKGGGGSEPKSSDKTHTGG | 49 |
| E200 + G4S | GGS | |
| linker + IgG | | |
| hinge + G4S linker | | |
| N-term extended | DFPGHNYTTRNILPGLNITSEPKSSDKTHT | 50 |
| E200 + IgG hinge | | |
| N-term extended | DFPGHNYTTRNILPGLNITSGSEPKSSDKTHT | 51 |
| E200 + GS | | |
| linker + IgG hinge | | |
| N-term extended | DFPGHNYTTRNILPGLNITSGGGGSEPKSSDKTHT | 52 |
| E200 + G4S | | |
| linker + IgG hinge | | |
| N and C term | DFPGHNYTTRNILPGLNITSTFHKTSGSGKEPKSSDKTHT | 53 |
| extended | | |
| E200 + IgG hinge | | |
| N and C term | DFPGHNYTTRNILPGLNITSTFHKTSGSGKGSEPKSSDKTHT | 54 |
| extended | | |
| E200 + GS | | |
| linker + IgG hinge | | |
| N and C term | DFPGHNYTTRNILPGLNITSTFHKTSGSGKGGGGSEPKSSDKTHT | 55 |
| extended E200 + | | |
| G4S linker + IgG | | |
| hinge | | |
| N-term extended | DFPGHNYTTRNILPGLNITSEPKSSDKTHTGS | 56 |
| E200 + IgG hinge + | | |
| GS linker | | |
| N-term extended | DFPGHNYTTRNILPGLNITSGSEPKSSDKTHTGS | 57 |
| E200 + GS | | |
| linker + IgG hinge + | | |
| GS linker | | |
| N-term extended | DFPGHNYTTRNILPGLNITSGGGGSEPKSSDKTHTGS | 58 |
| E200 + G4S | | |
| linker + IgG hinge + | | |
| GS linker | | |
| N-term and C | DFPGHNYTTRNILPGLNITSTFHKTSGSGKEPKSSDKTHTGS | 59 |
| term extended | | |
| E200 + IgG | | |
| hinge + GS linker | | |
| N-term and C | DFPGHNYTTRNILPGLNITSTFHKTSGSGKGSEPKSSDKTHTGS | 60 |
| term extended | | |
| E200 (underlined) + | | |
| GS linker + IgG | | |
| hinge + GS linker | | |
| N-term and C | DFPGHNYTTRNILPGLNITSTFHKTSGSGKGGGGSEPKSSDKTHT | 61 |
| term extended | GS | |
| E200 + G4S | | |
| linker + IgG | | |
| hinge + GS linker | | |
| N-term extended | DFPGHNYTTRNILPGLNITSEPKSSDKTHTGGGGS | 62 |
| E200 + IgG hinge + | | |
| G4S linker | | |
| N-term extended | DFPGHNYTTRNILPGLNITSGSEPKSSDKTHTGGGGS | 63 |
| E200 + GS | | |
| linker + IgG hinge + | | |
| G4S linker | | |
| N-term extended | DFPGHNYTTRNILPGLNITSGGGGSEPKSSDKTHTGGGGS | 64 |
| E200 + G4S | | |
| linker + IgG hinge + | | |
| G4S linker | | |
| N-term and C | DFPGHNYTTRNILPGLNITSTFHKTSGSGKEPKSSDKTHTGGGGS | 65 |
| term extended | | |
| E200 + IgG | | |
| hinge + G4S linker | | |
| N-term and C | DFPGHNYTTRNILPGLNITSTFHKTSGSGKGSEPKSSDKTHTGGG | 66 |
| term extended | GS | |
| E200 + GS linker + | | |
| lgG hinge + G4S | | |
| linker | | |
| N-term and C | DFPGHNYTTRNILPGLNITSTFHKTSGSGKGGGGSEPKSSDKTHT | 67 |
| term extended | GGGGS | |
| E200 + G4S | | |
| linker + IgG | | |
| hinge + G4S linker | | |
| “Native peptide 1” | GHNYTTRNILPGLNITSTFHKTSGSGKGGGGS | 68 |
| E200 underlined | | |
| “Native peptide 3” | GGGGSGHNYTTRNILPGLNITSTFHKTSGSGK | 69 |
| C-term extended | | |
| E200 underlined | | |
| “Native peptide 4” | GGGGSDFPGHNYTTRNILPGLNITSTFHKTSGSGK | 70 |
| N and C term | | |
| Extended E200 | | |
| underlined | | |

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

One aspect of the invention described herein is based on the identification that it is possible to reduce the capacity of a cellular immunotherapeutic (e.g. CAR T cell), expressing a receptor that binds to a dysfunctional P2X₇ receptor, to bind to the dysfunctional P2X₇ receptor on the surface of a cancer cell. The inventors have determined that this can be accomplished by providing a polypeptide that competes with the dysfunctional P2X₇ receptor for binding to the cellular immunotherapeutic. For example, the in vivo activity of a T cell expressing a CAR that comprises an antigen binding domain that binds to a dysfunctional P2X₇ receptor can be reduced by administering a polypeptide that comprises the same epitope of a dysfunctional P2X₇ receptor to which the antigen binding domain of the CAR binds. This reduction or inhibition is reversible and the viability of the cellular immunotherapeutic can be maintained.

Another aspect of the invention described herein, results in irreversible inhibition or reduction of in vivo activity of a cellular immunotherapeutic by targeting the cellular immunotherapeutic for cell killing. The targeting is accomplished by administering a polypeptide that comprises (a) an epitope of a dysfunctional P2X₇ receptor to which the cellular immunotherapeutic can bind and (b) a sequence for facilitating targeting of the cellular immunotherapeutic for cell-mediated killing. For example, the in vivo activity of a T cell expressing a CAR that comprises an antigen binding domain that binds to a dysfunctional P2X₇ receptor can be irreversibly reduced by administering a polypeptide that comprises (a) the same epitope of a dysfunctional P2X₇ receptor to which the antigen binding domain of the CAR binds, and (b) a sequence (e.g. an Fc region) that targets the CAR T cell for cell mediated killing (e.g. cytotoxic T cell or NK cell mediated killing).

### Definitions

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

“Antibodies” or “immunoglobulins” or “Igs” are gamma globulin proteins that are found in blood, or other bodily fluids of vertebrates that function in the immune system to bind antigen, hence identifying and/or neutralising foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (CH) for each of the α and γ chains and four CH domains for μ and ε isotypes. Each L chain has at the N-terminus, a variable domain (V_{L}) followed by a constant domain (CL) at its other end. The V_{L} is aligned with the V_{H} and the C_{L} is aligned with the first constant domain of the heavy chain (CHL).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in ¾ sequence and function, e.g., humans express the following subclasses: IgG1, IgG2, IgG3, IgG4, IgAI, and IgA2. The L chain from any vertebrate species can be assigned to one of two clearly distinct types, called kappa and lambda, based on the amino acid sequences of their constant domains.

The constant domain includes the Fc portion that comprises the carboxy-terminal portions of both H chains held together by disulfides. The effector functions of antibodies such as ADCC are determined by sequences in the Fc region, which region is also the part recognised by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” that affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

“Hypervariable region” refers to the regions of an antibody variable domain that are hypervariable in sequence and/or form structurally defined loops. Generally, antibodies comprise six hypervariable regions; three in the V_{H} (H1, H2, H3), and three in the V_{L} (L1, L2, L3).

“Framework” or “FR” residues are those variable domain residues other than the hypervariable region residues herein defined.

An “antigen binding site” generally refers to a molecule that includes at least the hypervariable and framework regions that are required for imparting antigen binding function to a V domain. An antigen binding site may be in the form of an antibody or an antibody fragment, (such as a mAb, single domain (SD)-mAb, dAb, Fab, SD-Fab, Fd, SD-Fv, Fv, F(ab′)2 or scFv) in a method described herein.

An “intact” or “whole” antibody is one that comprises an antigen-binding site as well as a C_{L} and at least heavy chain constant domains, CH1, CH2 and CH3. The constant domains may be native sequence constant domains (e.g. human native sequence constant domains) or amino acid sequence variant thereof.

“Whole antibody fragments including a variable domain” include SD-mAb, Fab, Fab′, F(ab′)2, and Fv fragments; diabodies; linear antibodies, single-chain antibody molecules; and multi-specific antibodies formed from antibody fragments.

The “Fab fragment” consists of an entire L chain along with the variable region domain of the H chain (V_{H}), and the first constant domain of one heavy chain (CHI). Each Fab fragment is monovalent with respect to antigen binding, i.e., it has a single antigen-binding site.

A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A “F(ab′)2 fragment” roughly corresponds to two disulphide linked Fab fragments having divalent antigen-binding activity and is still capable of cross-linking antigen.

An “Fv” is the minimum antibody fragment that contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains which enables the scFv to form the desired structure for antigen binding.

A “single variable domain” is half of an Fv (comprising only three CDRs specific for an antigen) that has the ability to recognise and bind antigen, although generally at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

An “isolated antibody” is one that has been identified and separated and/or recovered from a component of its pre-existing environment. Contaminant components are materials that would interfere with therapeutic uses for the antibody, and may include enzymes, hormones, and other proteinaceous or non-proteinaceous solutes.

A “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanised’ forms of non-human (e.g., rodent) antibodies are chimeric antibodies that contain minimal sequence derived from the non-human antibody. For the most part, humanised antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesised uncontaminated by other antibodies. Monoclonal antibodies may be prepared by the hybridoma methodology. The “monoclonal antibodies” may also be isolated from phage antibody libraries using molecular engineering techniques.

As used herein “tumour-associated antigen” refers to an antigen that is expressed by cancer cells (the term “tumour-antigen” may also be used to refer to same). Tumour antigens are proteins that are produced by tumour cells that elicit an immune response, particularly T-cell mediated immune responses. Tumour antigens are well known in the art and include, for example, a glioma-associated antigen, carcinoembryonic antigen (CEA), β-human chorionic gonadotropin, alpha fetoprotein (AFP), lectin-reactive AFP, thyroglobulin RAGE-1, MN-CAIX, human telomerase reverse transcriptase, RU1, RU2 (AS), intestinal carboxyl esterase, mut hsp70-2, M-CSF, hK4 prostase, prostate-specific antigen (PSA), PAP, NY-ESO-1, LAGE-1a, p53, P501S prostein, PSMA, Her2/neu, survivin and telomerase, prostate-carcinoma tumour antigen-1 (PCTA-1), MAGE, ELF2M, neutrophil elastase, ephrinB2, CD22, insulin growth factor (IGF)-I, IGF-II, IGF-I receptor and mesothelin,

In one embodiment, the tumour antigen comprises one or more antigenic cancer epitopes associated with a malignant tumour. Malignant tumours express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-foetal antigens such as carcinoembryonic antigen (CEA). In B-cell lymphoma the tumour-specific idiotype immunoglobulin constitutes a truly tumour-specific immunoglobulin antigen that is unique to the individual tumour. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success. The type of tumour antigen referred to in the invention may also be a tumour-specific antigen (TSA). A TSA is unique to tumour cells and does not occur on other cells in the body. A tumour-associated antigen (TAA) is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells. Those tumour-associated antigens of greatest clinical interest are differentially expressed compared to the corresponding non-tumour tissue and allow for a preferential recognition of tumour cells by specific T-cells or immunoglobulins.

Non-limiting examples of TSA or TAA antigens include the following: Differentiation antigens such as MART-1/MelanA (MART-1), gp 100 (Pmel 17), tyrosinase, TRP-1, TRP-2 and tumour-specific multilineage antigens such as MAGE-1, MAGE-3, BAGE, GAGE-1, GAGE-2, p15; overexpressed embryonic antigens such as CEA; overexpressed oncogenes and mutated tumour-suppressor genes such as p53, Ras, HER-2/neu; unique tumour antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 180erbB-3, c-met, nm-23H 1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA195, CA242, CA-50, CAM43, CD68\P 1, CO-029, FGF-5, G250, Ga733\EpCAM, HTgp-175, M344, MA-50, MG7-Ag, MOV 18, NB/70K, NY-CO-1, RCAS 1, SDCCAG16, TA-90\Mac-2 binding protein\cyclophilin C-associated protein, TAAL6, TAG72, TLP, and TPS. Particularly preferred examples of target cell antigens in accordance with the present invention include: CD33 (Siglec-3), CD123 (IL3RA), CD135 (FLT-3), CD44 (HCAM), CD44V6, CD47, CD184 (CXCR4), CLEC12A (CLL1), LeY, FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, PCSA CD276 and dysfunctional (nf)P2X₇ receptor.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 below:

SEQ ID NO: 1
MPACCSCSDVFQYETNKVTRIQSMNYGTIKWFFHVIIFSYVCFALVSDK
LYQRKEPVISSVHTKVKGIAEVKEEIVENGVKKLVHSVFDTADYTFPLQ
GNSFFVMTNFLKTEGQEQRLCPEYPTRRTLCSSDRGCKKGWMDPQSKGI
QTGRCVVYEGNQKTCEVSAWCPIEAVEEAPRPALLNSAENFTVLIKNNI
DFPGHNYTTRNILPGLNITCTFHKTQNPQCPIFRLGDIFRETGDNFSDV
AIQGGIMGIEIYWDCNLDRWFHHCRPKYSFRRLDDKTTNVSLYPGYNFR
YAKYYKENNVEKRTLIKVFGIRFDILVFGTGGKFDIIQLVVYIGSTLSY
FGLAAVFIDFLIDTYSSNCCRSHIYPWCKCCQPCVVNEYYYRKKCESIV
SPVWCQCGSCLPSQLPESHRCLEELCCRKKPGACITTSELFRKLVLSRH
VEPKPTLKYVSFVDESHIRMVNQQLLGRSLQDVKGQEVPRPAMDFTDLS
RLPLALHDTPPIPGQPEEIQLLRKEATPRSRDLQFLLLYQEPLLALDVD
STNSRLRHCAYRCYATWRFGSQDMADFAILPSCCRWRIRKEFPKSEGQY
SGFKSPY

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial, mesenchymal, germinal, neural and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTNILPGLNITC (SEQ ID NOs: 2-11; 15-70).

“E300 epitope” generally refers to an epitope having the sequence KYYKENNVEKRTLIK (SEQ ID NO: 12 and 13).

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 14).

The term “anti-P2X₇ receptor antibody” or “an antibody that binds to P2X₇ receptor” refers to an antibody that is capable of binding P2X₇ receptor with sufficient affinity such that the antibody is useful as a diagnostic and/or therapeutic agent in targeting P2X₇ receptor, typically non-functional P2X₇ receptor or a cancer associated P2X₇ receptor. Preferably, the extent of binding of a P2X₇ receptor antibody to an unrelated protein is less than about 10% of the binding of the antibody to P2X₇ receptor as measured, e.g., by a radioimmunoassay (RIA), Enzyme-Linked Immunosorbent Assay (ELISA), Biacore or Flow Cytometry. In certain embodiments, an antibody that binds to P2X₇ receptor has a dissociation constant (Kd) of <1 μM, <100 nM, <10 nM, <1 nM, or <0.1 nM. An anti nfP2X₇ receptor antibody is generally one having some or all of these serological characteristics and that binds to dysfunctional P2X₇ receptors but not to functional P2X₇ receptors.

An “affinity matured’ antibody is one with one or more alterations in one or more hypervariable regions thereof that result in an improvement in the affinity of the antibody for the antigen, compared to a parent antibody that does not possess those alteration(s). Preferred affinity matured antibodies will have nanomolar or even picomolar affinities for the target antigen. Affinity matured antibodies are produced by procedures known in the art.

A “blocking” antibody” or an “antagonist” antibody is one that inhibits or reduces biological activity of the antigen it binds. Preferred blocking antibodies or antagonist antibodies substantially or completely inhibit the biological activity of the antigen.

An “agonist antibody”, as used herein, is an antibody, which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The term “disorder” or “condition” means a functional abnormality or disturbance in a subject such as a cancer, an autoimmune disorder, or an infection by virus, bacteria, parasite, or others.

For example, a nucleic acid or a peptide naturally present in a living animal is not “isolated”, but the same nucleic acid or peptide partially or completely separated from the coexisting materials of its natural state is “isolated”. An isolated nucleic acid or protein can also exist in a non-native environment such as, for example, in a host cell.

As used herein, the term “subject” refers to a mammal such as mouse, rat, cow, pig, goat, chicken, dog, monkey or human. Preferentially, the subject is a human. The subject may be a subject suffering from a disorder such as cancer (a patient), but the subject also may be a healthy subject. As used herein, the terms “subject”, “individual” and “patient” may be used interchangeable.

The term “autologous” as used herein refers to any material derived from the same subject to whom it is later re-introduced.

The term “allogeneic” as used herein refers to any material derived from a different subject of the same species as the subject to whom the material is re-introduced.

The terms “therapeutically effective amount” or “therapeutically effective population” mean an amount of, for example, a cell population that provides a therapeutic benefit in a subject.

The terms “binds to”, “specifically binds to” or “specific for” with respect to a targeting moiety, as used e.g. of a CAR referring to an antigen-binding domain that recognises and binds to a specific antigen, does not substantially recognise or bind to other molecules in a sample. An antigen-binding domain or targeting moiety that binds specifically to an antigen from one species also may bind to that antigen from another species. This cross-species reactivity is typical of many antibodies and therefore not contrary to the definition that the antigen-binding domain is specific. An antigen-binding domain that specifically binds to an antigen may bind also to different allelic forms of the antigen (allelic variants, splice variants, isoforms etc.) or homologous variants of this antigen from the same gene family. This cross reactivity is typical of many antibodies and therefore not contrary to the definition that the antigen-binding domain is specific.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

The term “treat” (treatment of) a disorder as used herein means to reduce the frequency or severity of at least one sign or symptom of a disease or disorder experienced by a subject.

The term “prevent” as used herein, is intended to refer to at least the reduction of likelihood of the risk of (or susceptibility to) acquiring a disease or disorder (i.e., causing at least one of the clinical symptoms of the disease not to develop in a individual that may be exposed to or predisposed to the disease but does not yet experience or display symptoms of the disease). Biological and physiological parameters for identifying such patients are provided herein and are also well known by physicians. For example, prevention of an aberrant immune response, may be characterised by an absence of an increased release of cytokines following treatment with a cellular immunotherapeutic agent.

The term “expression” as used herein is defined as the transcription and/or translation of a particular nucleotide sequence driven by its promoter in a cell.

### Cellular Immunotherapeutic

As described herein, the invention includes the use of a molecule for binding to a cellular immunotherapeutic, wherein the cellular immunotherapeutic comprises immune cells that express a receptor comprising an antigen-recognition domain, and preferably a signaling domain, wherein the antigen-recognition domain recognises a target antigen. Preferably the target antigen is expressed on a cell surface and is a tumour-associated or tumour-specific antigen as further defined herein.

The skilled person will appreciate that the methods of the invention can be applied to a wide variety of different cellular immunotherapeutics. In certain embodiments, the cellular immunotherapeutic comprises an immune cell that expresses a chimeric antigen receptor (CAR) (or variant thereof including a ligand-based CAR) or a modified T cell Receptor (modified TCR), whereby the immune cell is a CAR, TCR, or variants thereof. It will further be appreciated that in preferred embodiments, the antigen-recognition domain will typically bind directly to a target antigen on a target cell (e.g., so-called “direct CARs”). However, the methods of the present invention can be applied in the context of cellular immunotherapeutics that interact indirectly with the target cell (e.g., via the binding to a ligand that facilitates the interaction with the target antigen on the target cell). Examples of such indirect cellular immunotherapeutics are reviewed in Arndt et al., (2020) Cancers (Basel), 12: 1302, incorporated herein by reference.

The cell may be an “engineered cell”, “genetically modified cell”, “immune cell” or “immune effector cell” as described herein. Further, the cell may be capable of differentiating into an immune cell. A cell that is capable of differentiating into an immune cell (e.g. T cell that will express a CAR) may be a stem cell, multi-lineage progenitor cell or induced pluripotent stem cell.

The immune cell of the invention can be any suitable immune cell, or progenitor cell thereof, or can be a homogeneous or a heterogeneous cell population. In some embodiments, the cell is a leukocyte, a Peripheral Blood Mononuclear Cell (PBMC), a lymphocyte, a T cell, a CD4+ T cell, a CD8+ T cell, a natural killer cell, a natural killer T cell, or a γδ T cell.

In any embodiment, the cell may be a T cell, wherein optionally said T cell does not express TcRαβ, PD1, CD3 or CD96 (e.g. by way of knocking down or knocking out one of these genes on a genetic level or functional level).

In any embodiment, the cell may be an immune cell, wherein optionally said cell does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the genetically modified cell includes two or more different CARs or different TCRs. As used herein, the term “different CARs” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of a co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

In general, a CAR or modified TCR may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide.

The extracellular domain of the antigen-recognition domain preferably recognises a target antigen expressed on a cancer cell. It will be appreciated that any number of different cellular immunotherapeutics expressing different antigen-recognition domains for binding different target antigens may be utilised in accordance with the present invention, although it will be necessary to utilise a molecule comprising an epitope that competes for binding to the cellular immunotherapeutic.

For example, the cellular immunotherapeutic may comprise a receptor with an antigen-recognition domain for binding to any one of: CD33 (Siglec-3), CD123 (IL3RA), CD135 (FLT-3), CD44 (HCAM), CD44V6, CD47, CD184 (CXCR4), CLEC12A (CLL1), FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, CD276 and PCSA.

In certain embodiments, the cellular immunotherapeutic is an immune cell expressing a CAR (or variant thereof) for binding dysfunctional P2X₇ receptor. The extracellular part of the CAR or variant thereof may comprise an nfP2X₇ binding domain that recognises the E200 (or E300 or E200-300 composite) epitope as disclosed herein.

Typically, the antigen-recognition domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor.

In such embodiments, the binding polypeptide comprises the amino acid sequence of the CDRs of the V_{H} and/or V_{L} chain of an antibody described in any one of: PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of the CDRs of the V_{H} and/or V_{L} chain of antibody 2-2-1 described in PCT/AU2010/001070 (or in any one of the corresponding U.S. Pat. No. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide of the CAR may comprise the amino acid sequences of the CDRs of the antibody sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

The binding polypeptide of the CAR may comprise the amino acid sequence of the V_{H} and/or V_{L} chains of an antibody described in any one of: PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. No. 8,440,186, 9,181,320, 9,944,701 or 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of the V_{H} and/or V_{L} chains of the antibody 2-2-1 described in PCT/AU2010/001070 (or in any one of the corresponding U.S. Pat. No. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide of the CAR may comprise the amino acid sequences of the V_{H} and/or V_{L} chains of the antibody sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

The binding polypeptide of the CAR may comprise the amino acid sequence of an antibody or fragment thereof described in any one of: PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. No. 8,440,186, 9,181,320, 9,944,701 or 10,597,451), PCT/AU2008/001365 (or in any one of the corresponding U.S. Pat. No. 8,293,491 or 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. No. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of sdAb 2-2-1 described in PCT/AU2010/001070 (or in any one of the corresponding U.S. Pat. No. 9,127,059, 9,688,771, or 10,053,508) or antibody BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide may comprise the amino acid sequences of sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

A “signal peptide” refers to a peptide sequence that directs the transport and localisation of the protein within a cell, e.g. to a certain cell organelle (such as the endoplasmic reticulum) and/or the cell surface.

Generally, an “antigen binding domain” refers to the region of the CAR that specifically binds to an antigen (and thereby is able to target a cell containing the antigen). The CARs of the invention may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanised antibody or antigen binding fragment thereof. Human or humanised antibodies or antigen binding fragments thereof can be made by a variety of methods well known in the art. The CAR as disclosed herein has an extracellular linker/label epitope binding domain as an antigen binding domain allowing it to bind indirectly via a target cell binding molecule as disclosed herein to an antigen expressed on a target cell.

“Spacer” or “hinge” as used herein refers to the hydrophilic region that is between the antigen binding domain and the transmembrane domain. The CARs of the invention may comprise an extracellular spacer domain but it is also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350: 293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Prominent examples of intracellular signalling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signalling sequences, firstly those that initiate antigen-dependent primary activation through the TCR (primary cytoplasmic signalling sequences) and secondly those that act in an antigen-independent manner to provide a secondary or co-stimulatory signal (secondary cytoplasmic signalling sequences, co-stimulatory signalling domain). Therefore, an intracellular signalling domain of a CAR may comprise one or more primary cytoplasmic signalling domains and/or one or more secondary cytoplasmic signalling domains.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

In some embodiments, the co-stimulatory receptor (from which a portion of signalling domain is derived) is selected from the group consisting of CD27, CD28, CD-30, CD40, DAP10, OX40, 4-1BB (CD137) and ICOS.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C and B7-H3.

The cytoplasmic signalling sequences within the cytoplasmic signalling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

The CAR of the present invention, i.e. the CAR comprising an nfP2X₇ E200 binding domain, may be designed to comprise any portion or part of the above-mentioned domains as described herein in any order and/or combination resulting in a functional CAR.

The CARs as disclosed herein, or polypeptide(s) derived therefrom, nucleic acid molecule(s) or recombinant expression vectors cells encoding said CARs, or populations of cells expressing said CARs, may be isolated and/or purified. The term “isolated” means altered or removed from the natural state. For example, an isolated population of cells means an enrichment of such cells and separation from other cells that are normally associated in their naturally occurring state with said isolated cells. An isolated population of cells means a population of substantially purified cells that are a more homogenous population of cells than found in nature. Preferably, the enriched cell population comprises at least about 90% of the selected cell type. In particular aspects, the cell population comprises at least about 90%, 91%, 92%, 93%, 94%, 95%, 96%, 97%, 98%, 99%, or even 100% of the selected cell type.

### Target Antigen Epitope

The present invention provides for the use of a molecule (for example a peptide or polypeptide) that comprises or consists of an epitope of a target antigen. The target cell antigen is recognised by the cellular immunotherapeutic and accordingly the molecule or polypeptide competes with the target antigen on a cancer cell for binding to the cellular immunotherapeutic.

As used herein, the terms peptide and polypeptide may be used interchangeably, particularly when the overall length of the molecule is less than 50 amino acids.

Typically, the epitope comprised in the molecule (for example, peptide or polypeptide) will comprise the same, or substantially the same amino acid sequence of the epitope for which the cellular immunotherapeutic is intended to bind on the target cell. For example, where the amino acid sequence of the epitope comprised in the molecule differs from the amino acid sequence comprised in the antigen on the target cell, the difference in amino acid sequence will not substantially impact on the ability of the molecule to bind to the cellular immunotherapeutic (or will still compete with the target antigen on the target cell for binding to the cellular immunotherapeutic). The amino acid sequence of the epitope comprised in the molecule may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of the epitope on the target antigen on the cell. In certain embodiments, the amino acid sequence of the epitope on the target antigen is the same sequence as the epitope comprised on the molecule used to inhibit the activity of the cellular immunotherapeutic.

In certain examples, the cellular immunotherapeutic is for binding the extracellular domain of CD19 on a target cell. It will be appreciated that in such examples, the molecule for use in accordance with the first or second aspects of the invention will comprise the same epitope of the ECD of CD19 that is bound by the cellular immunotherapeutic. Cellular immunotherapeutics for targeting CD19 are known to the skilled person, as are the epitopes to which such immunotherapeutics bind. For example, for inhibiting the activity of CAR-T cells comprising a CAR that has an antigen-recognition domain consisting of anti-CD19 scFv FMC683, the skilled person will appreciate that the molecule for use in accordance with the first or second aspects of the invention should comprise an epitope that is bound by scFv FMC683. Similarly, for inhibiting the activity of CAR-T cells comprising a CAR that has an antigen-recognition domain consisting of anti-CD19 scFv A3B1, the skilled person will appreciate that the molecule for use in accordance with the first or second aspects of the invention should comprise an epitope that is bound by scFv A3B1. The epitopes bound by anti-CD19 antibodies FMC683, 3B110, and 4G7-2E3, which are used in various anti-CD19 cellular immunotherapeutics, are described in Klesmith et al., (2019) Biochemistry, 58:489-4881, incorporated herein by reference.

In a similar example, the cellular immunotherapeutic is for binding CD20 on a target cell. It will be appreciated that in such examples, the molecule for use in accordance with the first or second aspects of the invention will comprise the same epitope of CD20 that is bound by the cellular immunotherapeutic.

In other examples, the cellular immunotherapeutic is for binding mesothelin and therefore the molecule or polypeptide comprises an epitope of mesothelin.

In other examples, the cellular immunotherapeutic is for binding EGFR and therefore the molecule or polypeptide comprises an epitope of EGFR.

In other examples, the cellular immunotherapeutic is for binding GPC3 and therefore the molecule or polypeptide comprises an epitope of GPC3.

In other examples, the cellular immunotherapeutic is for binding MUC1 and therefore the molecule or polypeptide comprises an epitope of the MUC1.

In other examples, the cellular immunotherapeutic is for binding HER2 and therefore the molecule or polypeptide comprises an epitope of HER2.

In other examples, the cellular immunotherapeutic is for GD2 and therefore the molecule or polypeptide comprises an epitope of GD2.

In other examples, the cellular immunotherapeutic is for binding CEA and therefore the molecule or polypeptide comprises an epitope of CEA.

In other examples, the cellular immunotherapeutic is for binding EpCAM and therefore the molecule or polypeptide comprises an epitope of EpCAM.

In other examples, the cellular immunotherapeutic is for binding LeY and therefore the molecule or polypeptide comprises an epitope of LeY.

In other examples, the cellular immunotherapeutic is for PSCA and therefore the molecule or polypeptide comprises an epitope of PCSA.

In other examples, the cellular immunotherapeutic is for CD276 and therefore the molecule or polypeptide comprises an epitope of CD276.

In other examples, the cellular immunotherapeutic is for binding dysfunctional P2X₇ receptor and therefore the molecule or polypeptide comprises an epitope of the dysfunctional P2X₇ receptor.

Typically, the epitope of the dysfunctional P2X₇ receptor comprises a peptide fragment of a dysfunctional P2X₇ receptor, wherein the fragment comprises an epitope that is not found on a functional P2X₇ receptor.

As such, a dysfunctional P2X₇ receptor epitope may be provided in the form of a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

A range of peptide fragments of a dysfunctional P2X₇ receptor are known and discussed in PCT/AU2002/000061 (and in corresponding publications WO 2002/057306 and U.S. Pat. No. 7,326,415, 7,888,473, 7,531,171, 8,080,635, 8,399,617, 8,709,425, 9,663,584, or 10,450,380), PCT/AU2008/001364 (and in corresponding publications WO 2009/033233 and U.S. Pat. No. 8,440,186, 9,181,320, 9,944,701 or 10,597,45) and PCT/AU2009/000869 (and in corresponding publications WO 2010/000041 and U.S. Pat. No. 8,597,643, 9,328,155 or 10,238,716) the contents of all of which are incorporated in entirety. Exemplary peptides within these specifications which include epitopes contemplated for use in this invention are described below.

| PCT publication | Peptide sequence |
|---|---|
| WO 2002/057306 | GHNYTTRNILPGLNITC (SEQ ID NO: 2) (also referred to |
| | herein as the “E200” epitope) |
| WO 2009/033233 | KYYKENNVEKRTLIKVF (SEQ ID NO: 3) (also referred to |
| | herein as the “E300” epitope) |
| WO 2010/000041 | GHNYTTRNILPGAGAKYYKENNVEK (SEQ ID NO: 4) (also |
| | referred to herein as the “E200/E300” or “composite” |
| | epitope). |

Additional examples of the E200, E300 and composite epitopes are described herein in Table 1. As discussed elsewhere herein, the peptide or polypeptide comprising the E200, E300 or composite epitope may contain additional amino acids. In certain examples, the additional amino acids may be derived directly from the adjacent sequences present within the nfP2X₇ receptor sequence. Alternatively, the additional amino acid sequences may be derived from one or more linker sequences, such as glycine/serine rich linker sequences, and/or hinge regions derived from immunoglobulins. The skilled person will appreciate that such modifications may be made to the E200, E300 or composite epitopes for the purposes of increasing the stability or solubility of the peptide or polypeptide, or for modulating the degree of binding to the CAR.

In any embodiment of the first or second aspects, the polypeptide reduces the ability of the cellular immunotherapeutic to bind to a dysfunctional P2X₇ receptor on a cell, preferably a cancer cell. Preferably the polypeptide blocks, or disrupts with the interaction of the cellular immunotherapeutic with the dysfunctional P2X₇ receptor present on a cancer cell.

In any embodiment of the first or second aspects, the epitope on the polypeptide comprises an amino acid sequences that is substantially the same, or homologous to the epitope on the dysfunctional P2X₇ receptor bound by the cellular immunotherapeutic. In other words, even though the amino acid sequence of the two epitopes may differ, there is sufficient homology for the cellular immunotherapeutic to bind to both the polypeptide and the dysfunctional P2X₇ receptor on a cell. In any embodiment, the epitope on the polypeptide comprises or consists of the amino acid sequence of the epitope on the dysfunctional P2X₇ receptor to which the cellular immunotherapeutic binds.

Molecules for Use in Accordance with the Invention

It will be appreciated that the architecture of the molecules of the invention will depend on whether the molecules are to be used in accordance with the first or second aspects of the invention.

In particular embodiments, the molecules may be in the form of polypeptides. Such polypeptides may be in the form of fusion proteins or chimeric proteins.

More specifically, the molecule will preferably be in the form of a polypeptide that comprises a sequence and architecture that facilitates a temporary and reversible effect on the cellular immunotherapeutic.

Taking the example of CAR-T cells for binding the target antigen, the polypeptide may comprise an epitope for binding to the antigen-recognition domain of the CAR, and may or may not comprise additional sequences for facilitating increased solubility and stability of the polypeptide.

In the particular example of anti-nfP2X₇ receptor CAR T cells, the molecules for use in the methods of the invention will preferably be in the form of a peptide or polypeptide, comprising the amino acid sequence of the E200, E300, or composite epitopes, as herein defined. In particularly preferred embodiments, the molecule is a polypeptide or peptide comprising the amino acid sequence of the E200 epitope, preferably comprising at least the sequence as set forth in any of SEQ ID NOs: 3, 4, 5 or 6. Additionally, the peptide or polypeptide may comprise additional amino acid residues derived from the nfP2X₇ receptor sequence and which occurs adjacently to the E200 epitope within the native receptor sequence. Further still, the polypeptide may comprise additional amino acid residues to improve the solubility and/or stability of the polypeptide, such as the addition of amino acid residues derived from glycine/serine rich linker regions and/or IgG hinge regions. Several examples of such additions to the “minimum” E200 epitope sequence are provided herein in Table 1 although the skilled person will appreciate that any number of alternative modifications can be used.

It will also be well within the purview of the skilled person to confirm the ability of a given molecule to be bound by the relevant CAR. For example in the context of nfP2X₇ receptor-binding CARs, the skilled person will be able to use routine techniques to confirm binding of the polypeptide (or series of polypeptides) by the CAR, and thereby determine the suitability of the polypeptide for use in methods of the invention.

In the broader context of CARs other than those designed for binding to nfP2X₇ receptor or to E200 epitope, the skilled person will similarly be able to determine whether the designed molecule or polypeptide is capable of being bound by the CAR. For example, having determined the immunotherapeutic to be inhibited, the skilled person can readily determine the antigen that the immunotherapeutic is designed to bind to. In the simplest example, this can be done by reference to the product information relating to the immunotherapeutic and knowledge of the target antigen. The skilled person can then formulate the amino acid sequence of the molecule (preferably peptide or polypeptide) for disrupting the interaction of the cellular immunotherapeutic and the target antigen. In the simplest example, the skilled person can design a peptide or polypeptide comprising the same amino acid sequence as the target antigen of the cellular immunotherapeutic.

Finally, the skilled person, having identified the appropriate amino acid content of the molecule for disrupting the interaction of the cellular immunotherapeutic and the target antigen of the invention, can readily determine, using routine techniques, whether the molecule: a) is bound by the CAR T cell and b) can successfully inhibit cell killing by the CAR T cell. Methods of determining binding to target antigens, and cytotoxicity are well known in the art. Non-limiting methods and various experimental protocols for determining binding to CAR T cell and reduction of cell killing are described in detail herein in the Examples.

Further examples of sequences for facilitating increased solubility and stability of the polypeptide may include a sequence of a serum albumin (preferably HSA), transferrin, a sequence of an Fc region of an antibody, or carboxy-terminal peptide of chorionic gonadotropin (CG) β chain. Other sequences may include non-structured polypeptide sequences for increasing the overall size and hydrodynamic radius of the overall polypeptide. Such approaches have previously been referred to as “XTENylation” (a non-exact repeat peptide sequence) “PASylation” (fusion of polypeptide sequences composed of proline-alanine-serine polymer, i.e., PAS), “ELPylation” (fusion to elastin-like peptide (ELP) repeat sequence), “HAPylation” (e.g., inclusion of a homopolymer of glycine residues) or GLK (gelatin-like protein) fusions.

It will be appreciated that a wide variety of other architectures could be employed, for example, the polypeptide comprising the epitope may be conjugated or fused to any suitable carrier moiety. In certain embodiments, the carrier moiety may be selected from: a carbohydrate, a lipid, a liposome, a peptide, and an aptamer.

In some embodiments, the polypeptide may be provided in the context of a liposome (e.g., a pegylated liposome) comprising the polypeptide on the surface of PEG couplings. This provides for a liposome that is coated in the epitope that is recognised and bound by the cellular immunotherapeutic.

In circumstances where the polypeptide comprises a sequence of an Fc region of an antibody, and in accordance with the first aspect of the invention, the Fc region will preferably not comprise a sequence that would otherwise target the cellular immunotherapeutic for cell killing, when bound by the polypeptide. In contrast, the polypeptide for use in accordance with the second aspect of the invention will preferably comprise an Fc region of an antibody that does comprise such a sequence.

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains.

In accordance with the first aspect of the invention, the Fc fusion protein preferably does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 Al).

Fc regions of antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Other Fc modifications for use in accordance with the first aspect of the invention include variants that reduce or ablate binding to FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region and for tuning the selectivity of the fusion proteins. Preferred knockout variants are described in US 2008-0242845 A1, published on Oct. 2, 2008, entitled “Fc Variants with Optimized Properties, expressly incorporated by reference herein. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in U.S. Ser. No. 11/256,060. Other modifications for reducing FcγR and complement interactions include but are not limited to substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691, incorporated by reference in its entirety.

Fc modifications that improve binding to FcγRs and/or complement may find use in accordance with the second aspect of the invention. Such Fc variants may enhance Fc-mediated effector functions such as ADCC, ADCP, and/or CDC. Preferred modifications for improving FcγR and complement binding are described in US 2006-0024298 A1, published on Feb. 2, 2006, and US 2006-0235208 A1, published on Oct. 19, 2006, expressly incorporated herein by reference. Preferred modifications comprise a substitution at a position selected from the group consisting of 236, 239, 268, 324, and 332, wherein numbering is according to the EU index. Preferred substitutions include but are not limited to 236A, 239D, 239E, 268D, 267E, 268E, 268F, 324T, 332D, and 332E. Preferred variants include but are not limited to 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 305I, and 396L. These and other modifications are reviewed in Strohl, 2009, ibid.

In one embodiment, the fusions disclosed herein may incorporate Fc variants that enhance affinity for an inhibitory receptor FcγRIIb. Such variants may provide the fusions herein with immunomodulatory activities related to FcγRIIb+ cells, including for example B cells and monocytes. In one embodiment, the Fc variants provide selectively enhanced affinity to FcγRIIb relative to one or more activating receptors. Modifications for altering binding to FcγRIIb are described in U.S. Ser. No. 12/156,183, filed May 30, 2008, entitled “Methods and Compositions for Inhibiting CD32b Expressing Cells”, herein expressly incorporated by reference. In particular, Fc variants that improve binding to FcγRIIb may include one or more modifications at a position selected from the group consisting of 234, 235, 236, 237, 239, 266, 267, 268, 325, 326, 327, 328, and 332, according to the EU index. Preferable substitutions for enhancing FcγRIIb affinity include but are not limited to 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. More preferably, substitutions include but are not limited to 235Y, 236D, 239D, 266M, 267E, 268D, 268E, 328F, 328W, and 328Y. Preferred Fc variants for enhancing binding to FcγRIIb include but are not limited to 235Y/267E, 236D/267E, 239D/268D, 239D/267E, 267E/268D, 267E/268E, and 267E/328F.

The fusion proteins described herein can incorporate Fc modifications in the context of any IgG isotype or IgG isotype Fc region, including but not limited to human IgG1, IgG2, IgG3, and/or IgG4. The IgG isotype may be selected such as to alter FcγR- and/or complement-mediated effector function(s). Hybrid IgG isotypes may also be useful. For example, U.S. Ser. No. 11/256,060 describes a number of hybrid IgG1/IgG2 constant regions that may find use in the particular invention. In some embodiments of the invention, the fusion proteins may comprise means for isotypic modifications, that is, modifications in a parent IgG to the amino acid type in an alternate IgG. For example, an IgG1/IgG3 hybrid variant may be constructed by a substitutional means for substituting IgG1 positions in the CH2 and/or CH3 region with the amino acids from IgG3 at positions where the two isotypes differ. Thus a hybrid variant IgG antibody may be constructed that comprises one or more substitutional means, e.g., 274Q, 276K, 300F, 339T, 356E, 358M, 384S, 392N, 397M, 422I, 435R, and 436F. In other embodiments of the invention, an IgG1/IgG2 hybrid variant may be constructed by a substitutional means for substituting IgG2 positions in the CH2 and/or CH3 region with amino acids from IgG1 at positions where the two isotypes differ. Thus a hybrid variant IgG antibody may be constructed that comprises one or more substitutional means, e.g., one or more of the following amino acid substitutions: 233E, 234L, 235L, −236G (referring to an insertion of a glycine at position 236), and 327A.

In embodiments of the second aspect of the invention, the fusion proteins disclosed herein may incorporate Fc variants that improve FcRn binding. Such variants may enhance the in vivo pharmacokinetic properties of the fusion proteins. Preferred variants that increase binding to FcRn and/or improve pharmacokinetic properties include but are not limited to substitutions at positions 259, 308, 428, and 434, including but not limited to for example 259I, 308F, 428L, 428M, 434S, 434H, 434F, 434Y, and 434M (U.S. Ser. No. 12/341,769, filed Dec. 22, 2008, entitled “Fc Variants with Altered Binding to FcRn”, entirely incorporated by reference). Other variants that increase Fc binding to FcRn include but are not limited to: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604, entirely incorporated by reference), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (dall Acqua et al. Journal of Immunology, 2002, 169:5171-5180, Dall'Acqua et al., 2006, Journal of Biological Chemistry 281:23514-23524, entirely incorporated by reference). Other modifications for modulating FcRn binding are described in Yeung et al., 2010, J Immunol, 182:7663-7671.

The polypeptide may be in the form of a fusion protein such that the region comprising the epitope and any further sequence are encoded by a single nucleic construct, i.e., a “genetic fusion”). Alternatively, the polypeptide may comprise the dysfunctional P2X₇ receptor epitope and any further sequence joined via chemical conjugation or non-covalent attachment via a peptide.

The fusion proteins described herein may comprise a linker region for linking the sequence comprising the dysfunctional P2X₇ receptor epitope and any further sequence as described herein (e.g., an Fc region of an antibody, an HSA sequence or the like).

The term “linker” is used to denote polypeptides comprising two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). A variety of linkers may find use in some embodiments described herein to covalently link two different polypeptide or peptide sequences.

“Linker” herein is also referred to as “linker sequence”, “spacer”, “tethering sequence” or grammatical equivalents thereof. Homo- or hetero-bifunctional linkers as are well known (see, 1994 Pierce Chemical Company catalogue, technical section on cross-linkers, pages 155-200, incorporated entirely by reference). A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used. Useful linkers include glycine-serine polymers, including for example (GS)n, (GSGGS (SEQ ID NO. 71))n, (GGGGS (SEQ ID NO. 72))n, and (GGGS (SEQ ID NO. 73))n, where n is an integer of at least one, glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers. The fusion proteins of the invention may comprise a linker region (or spacer) located between the dysfunctional P2X₇ receptor epitope and any further sequence as described herein.

A linker is usually a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids.

In some aspects, the fusion protein of the present invention includes a peptide linker. The skilled person will be familiar with the design and use of various peptide linkers comprised of various amino acids, and of various lengths, which would be suitable for use as linkers in accordance with the present invention. The linker may comprise various combinations of repeated amino acid sequences.

The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage). Examples of such linkers are known to the skilled person and are described for example, in Chen et al., (2013) Advanced Drug Delivery Reviews, 65:1357-1369.

In some aspects, the peptide linker may include the amino acids glycine and serine in various lengths and combinations. In some aspects, the peptide linker can include the sequence Gly-Gly-Ser (GGS), Gly-Gly-Gly-Ser (GGGS (SEQ ID NO. 73)) or Gly-Gly-Gly-Gly-Ser (GGGGS (SEQ ID NO. 72)) and variations or repeats thereof. In some aspects, the peptide linker can include the amino acid sequence GGGGS (SEQ ID NO. 72) (a linker of 6 amino acids in length) or even longer. The linker may a series of repeating glycine and serine residues (GS) of different lengths, i.e., (GS)ₙ where n is any number from 1 to 15 or more. For example, the linker may be (GS)₃ (i.e., GSGSGS (SEQ ID NO. 74)) or longer (GS)₁₁ or longer. It will be appreciated that n can be any number including 1, 2, 3, 4, 5, 6, 7, 8, 9, 10, 11 or more. Fusion proteins having linkers of such length are included within the scope of the present invention. Similarly, the linker may be a series of repeating glycine residues separated by serine residues. For example (GGGGS (SEQ ID NO. 72))₃ (i.e., the linker may comprise the amino acid sequence GGGGSGGGGSGGGGS (SEQ ID NO. 75) (G₄S)₃) and variations thereof.

The peptide linker may consist of a series of repeats of Thr-Pro (TP) comprising one or more additional amino acids N and C terminal to the repeat sequence. For example, the linker may comprise or consist of the sequence GTPTPTPTPTGEF (SEQ ID NO. 76) (also known as the TP5 linker). In further aspects, the linker may be a short and/or alpha-helical rigid linker (e.g. A(EAAAK)3A (SEQ ID NO. 77), PAPAP (SEQ ID NO. 78) or a dipeptide such as LE).

In certain aspects, the linker may be flexible and cleavable. Such linkers preferably comprise one or more recognition sites for a protease to enable cleavage.

Preferred linkers of the invention comprise sequences from an antibody hinge region. Hinge regions sequences from any antibody isotype may be used, including for example hinge sequences from IgG1, IgG2, IgG3, and/or IgG4. Linker sequences may also include any sequence of any length of CL/CH1 domain but not all residues of CL/CH1 domain; for example the first 5-12 amino acid residues of the CL/CH1 domains. Linkers can be derived from immunoglobulin heavy chains of any isotype, including for example Cγ1, Cγ2, Cγ3, Cγ4, Cα1, Cα2, Cδ, Cε, and Cμ. Linkers can be derived from immunoglobulin light chain, for example Cκ or Cλ. Linker sequences may also be derived from other proteins such as Ig-like proteins (e.g. TCR, FcR, KIR), hinge region-derived sequences, and other natural sequences from other proteins.

In alternative embodiments of the second aspect of the invention, the molecule may be in the form of a protein conjugate, which comprises a first moiety in the form of the epitope of the target antigen (i.e., for competing with binding to the cellular immunotherapeutic) and a second moiety in the form of a toxin. Preferably, upon binding of such a molecule to the cellular immunotherapeutic, the cellular immunotherapeutic undergoes cell death mediated by the toxin. It will be appreciated that any suitable toxin may be utilised (for example, such as those toxins that are typically conjugated to antibodies for eliciting cell death).

In certain embodiments of the second aspect of the invention, the molecule may be in the form of an anti-idiotypic antibody for binding to the cellular immunotherapeutic, and to which is conjugated a cytotoxin or chemotherapeutic for triggering cell death of the cellular immunotherapeutic.

The skilled person will be well able to identify suitable anti-idiotypic antibodies for use in accordance with such embodiments of the invention. Furthermore, the skilled person will be familiar with suitable cytotoxic agents for conjugating to the anti-idiotypic antibody for use in this aspect of the invention. Cytotoxic agents include, but are not limited to, radioactive isotopes (e.g., At211, 1131, 1125, Y90, Rel86, Rel88, Sml53, Bi212, P32, Pb212 and radioactive isotopes of Lu); chemotherapeutic agents (e.g., maytansinoids, auristatins, dolostatin, a calicheamicin or derivatives thereof, taxanes, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes. Also among the anti-idiotype antibody immunoconjugates are those in which the antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Conjugates of an anti-idiotype antibody and cytotoxic agent may be made using any of a number of known protein coupling agents, e.g., linkers, (see Vitetta et al., Science 238: 1098 (1987)), WO94/11026. The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52: 127-131 (1992); U.S. Pat. No. 5,208,020).

### Nucleic Acids

The present invention also provides a nucleic acid molecule encoding a polypeptide as described herein for use in inhibiting the activity of a cellular immunotherapeutic.

The nucleic acid may be utilised to generate the polypeptide intended for administration in accordance with the present invention, using in vitro methods. Alternatively, the nucleic acid may facilitate in vivo expression of the polypeptide such that the nucleic acid is administered to the subject requiring treatment. In such embodiments, the nucleic acid may comprise a nucleic acid sequence encoding the polypeptide, and a controllable promoter for inducing expression thereof. Alternatively, the promoter may provide for constitutive expression of the polypeptide. Further still, it will be appreciated that depending on the context, the nucleic acid may be for transient expression of the nucleic acid sequence encoding the polypeptide.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the second aspect of the invention, the nucleic acid molecule comprises a nucleic acid sequence encoding the amino acid sequence of any one of SEQ ID NOs: 2, 3 and 4.

Further, the present invention provides a nucleic acid construct including a nucleic acid molecule encoding a polypeptide of the invention, or part thereof. The nucleic acid construct may further comprise one or more of: an origin of replication for one or more hosts; a selectable marker gene that is active in one or more hosts; and/or one or more transcriptional control sequences.

As used herein, the term “selectable marker gene” includes any gene that confers a phenotype on a cell in which it is expressed, to facilitate the identification and/or selection of cells that are transfected or transformed with the construct.

“Selectable marker genes” include any nucleotide sequences which, when expressed by a cell transformed with the construct, confer a phenotype on the cell that facilitates the identification and/or selection of these transformed cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance genes; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence that effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid that confers, activates or enhances expression of a nucleic acid in a cell.

In some embodiments, at least one transcriptional control sequence is operably connected to the nucleic acid molecule of the second aspect of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter that is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken p actin (CAGG).

Inducible promoters may include, but are not limited to, chemically inducible promoters and physically inducible promoters. Chemically inducible promoters include promoters that have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

As mentioned above, the control sequences may also include a terminator. The term “terminator” refers to a DNA sequence at the end of a transcriptional unit that signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence that is operable in the cells, tissues or organs in which it is intended to be used. Suitable terminators would be known to a person skilled in the art.

As will be understood, the nucleic acid constructs of the invention can further include additional sequences, for example sequences that permit enhanced expression, cytoplasmic or membrane transportation, and location signals. Specific non-limiting examples include an Internal Ribosome Entry Site (IRES) or cleavage site (e.g. P2A, T2A).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid construct of the third aspect of the present invention, into eukaryotic cells. As will be understood, the method best suited for introducing the nucleic acid construct into the desired host cell is dependent on many factors, such as the size of the nucleic acid construct, the type of host cell, the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation, sonoporation, heat-shock or optical transfection; particle-based methods such as ‘gene gun’ delivery, magnetofection, or impalefection or viral transduction.

The nucleic acid construct will be selected depending on the desired method of transfection/transduction. In some embodiments of the third aspect of the invention, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000; 11: 2377-87) and more generally utilising retroviral systems for transduction of mammalian cells (Cepko, C. and Pear, W. Curr Protoc Mol Biol. 2001, unit 9.9). In other embodiments, the nucleic acid construct is a plasmid, a cosmid, an artificial chromosome or the like, and can be transfected into the cell by any suitable method known in the art.

### Methods of Treatment and Administration

As discussed further in this document, the present invention finds application in the treatment of a variety of conditions, although preferably in the treatment of cancers.

More specifically, the invention finds particular application in the fine-tuning or switching off of various cellular immunotherapies that are targeted to dysfunctional P2X₇ receptors. Such applications may find use in treating, minimising or reducing the risk of aberrant immune responses that results from such cellular immunotherapies.

Inappropriate or uncontrolled activation of the immune system can result in a potentially fatal immune reaction consisting of a positive feedback loop between cytokines and white blood cells, with highly elevated levels of various cytokines. Such uncontrolled immune activation, often called a cytokine cascade, cytokine-associated toxicity, cytokine release syndrome can be induced by a number of physical conditions or medical therapies, most notably immunotherapies which specifically exploit the immune system of the recipient to fight a disease.

Although different terms may be used to describe cytokine storm, cytokine cascade or cytokine release syndrome, all of these conditions have in common the uncontrolled activation of the immune system, which can lead to potentially fatal consequences.

As used herein, cytokine-associated toxicity refers to a potentially life-threatening adverse cytokine response to aberrant immune system activation, for example caused by illness but also including an immunomodulating therapy.

Cytokine-associated toxicity is also described in the art using the term cytokine release syndrome (CRS). When sufficiently severe, this syndrome can be referred to as hypercytokinemia or ‘cytokine storm’. As used herein, CRS defines a systemic inflammatory response in a patient inter alia characterised by hypotension, pyrexia and/or rigors, and potentially resulting in death. CRS is believed to be caused by an uncontrolled positive feedback loop between cytokines and immune cells, resulting in highly elevated levels of various cytokines. CRS also involves the systemic expression of immune system mediators and includes increased levels of pro-inflammatory cytokines and anti-inflammatory cytokines.

The skilled person will be familiar with the clinical manifestations of cytokine-associated toxicity or hypercytokinemia and therefore will also be familiar with various methods for identifying an individual in need of treatment for cytokine-associated toxicity including hypercytokinemia.

The below table lists some of the recognised clinical signs and symptoms of cytokine-associated toxicity:

TABLE 2
Clinical signs of cytokine-associated toxicity

| Organ | |
|---|---|
| system | Symptoms |
| Constitutional | Fever ± rigors, malaise, fatigue, anorexia, myalgia, |
| | arthralgia, nausea, vomiting, headache |
| Skin | Rash |
| Gastrointestinal | Nausea, vomiting, diarrhoea |
| Respiratory | Tachypnoea, hypoxaemia |
| Cardiovascular | Tachycardia, widened pulse pressure, hypotension, |
| | increased cardiac output (early), potentially diminished |
| | cardiac output (late) |
| Coagulation | Elevated D-dimer, hypofibrinogenaemia ± bleeding |
| Renal | Azotaemia |
| Hepatic | Transaminitis, hyperbilirubinaemia |
| Neurologic | Headache, mental status changes, confusion, delirium, |
| | word finding difficulty or frank aphasia, hallucinations, |
| | tremor, dymetria, altered gait, seizures |

In addition, the National Cancer Institute Common Terminology Criteria for Adverse Events (CTCAE v 4.0) contains a grading system designed for cytokine response syndrome associated with antibody therapeutics, as shown below:
Grade 1—Mild reaction; infusion interruption not indicated; intervention not indicated
Grade 2—Therapy or infusion interruption indicated but responds promptly to symptomatic treatment (e.g. antihistamines, NSAIDS, narcotics, IV fluids); prophylactic medications indicated for <24 hrs
Grade 3—Prolonged (e.g., not rapidly responsive to symptomatic medication and/or brief interruption of infusion); recurrence of symptoms following initial improvement; hospitalisation indicated for clinical sequelae (e.g., renal impairment, pulmonary infiltrates)
Grade 4—Life-threatening consequences; pressor or ventilatory support indicated

Hypercytokinemia, as employed herein is a potentially fatal immune response resulting from the inappropriate positive signalling between cytokines and immune cells and ultimately cytokine release. In patients this leads to a high fever, swelling and redness, extreme fatigue, nausea and in some instances is fatal. Whilst more than 150 known inflammatory mediators are thought to be released during cytokine storm, generally in the methods of the present invention, the skilled person can determine the level of cytokine-associated toxicity by measuring serum levels of one or more suitable cytokines, for example 1, 2, 3, 4, 5, 6, 7, 8, 9, 10 or more cytokines can be measured, such as cytokines independently selected from IL-Iβ, TNFα, IL-6, IL-8 (CXCL8), IL-2, IL-10, IFNy, IL-12p70 and GM-CSF (for example IL-6, TNFα, IFNy, IL-2 and IL-8).

Laboratory based methods for measuring serum levels of cytokines indicative of cytokine-associated toxicity will be known to the skilled person. These methods will be useful in the methods of the present invention, for example, for determining whether an individual is suffering from cytokine-associated toxicity and also to determine whether the treatment in accordance with the present invention has been successful.

A variety of methods for determining cytokine levels in biological samples, including serum and plasma samples are known to the skilled person. Briefly, the levels of inflammatory cytokines can be determines in biological samples by enzyme-linked immunosorbent assays (ELISAs) using ELISA kits according to manufacturer's protocols.

Alternatively, levels of serum cytokines can be determined using multiplex bead array kits in accordance with the manufacturer's instructions (for example, Bio-Plex Human Cytokine Assay).

The skilled person will also appreciate that baseline inflammatory cytokine levels can be very high in certain individuals, due to their underlying disease (for which they are receiving immunotherapy). Accordingly, the skilled person will also appreciate that determining fold increases, net increases or rate of change increases in cytokine levels may provide a better indication of the likelihood of cytokine-associated toxicity in an individual, rather than absolute levels.

Other methods of determining likelihood of cytokine-associated toxicity include monitoring of proteins, which are indicative of elevated cytokine levels. For example, C-reactive protein (CRP) is an acute phase protein produced by the liver and which can often serve as a reliable surrogate for IL-6 bioactivity. Accordingly, the skilled person will also appreciate the utility in measuring CRP levels as a means for identifying an individual in need of treatment for cytokine-associated toxicity.

The methods of the invention may also be used to dampen the activity of a cellular immunotherapeutic or permanently switch off the activity of the cellular immunotherapeutic in circumstances where tumour lysis syndrome is suspected or a risk.

Accordingly, the present invention provides a method for minimising or reducing the risk of tumour lysis syndrome in a subject who has received or is receiving a therapy with a cellular immunotherapeutic for binding to a target antigen on a cell (such as, but not limited to dysfunctional P2X₇ receptor), the method comprising:
providing a subject who has received or is receiving a therapy with a cellular immunotherapeutic for binding to a target antigen on a cell;
administering to the subject, a molecule, optionally a polypeptide for binding to the cellular immunotherapeutic or a nucleic acid encoding said polypeptide, the molecule or polypeptide comprising or consisting of an epitope of the target antigen;
   wherein the epitope on the molecule or polypeptide competes with an epitope on the target antigen for binding to the cellular immunotherapeutic and the molecule or polypeptide thereby disrupts the interaction between the cellular immunotherapeutic and the target antigen on the cell;
   thereby minimising or reducing the risk of tumour lysis syndrome in the subject. Preferably, the method does not reduce the viability of the cells of the cellular immunotherapeutic.

In a further embodiment, the present invention provides a method for treating tumour lysis syndrome in a subject who has received or is receiving a therapy with a cellular immunotherapeutic for binding to a target antigen (such as but not limited to dysfunctional P2X₇ receptor), the method comprising:
providing a subject who has received or is receiving a therapy with a cellular immunotherapeutic for binding to a target antigen on a cell;
administering to the subject, a molecule, optionally in the form of a polypeptide for binding to the cellular immunotherapeutic or a nucleic acid encoding said polypeptide, the molecule or polypeptide comprising or consisting of an epitope of the target antigen;
   wherein the epitope on the molecule polypeptide competes with an epitope on the target antigen for binding to the cellular immunotherapeutic and the molecule or polypeptide thereby disrupts the interaction between the cellular immunotherapeutic and the target antigen on the cell;
   thereby treating tumour lysis syndrome in the subject. Preferably, the method does not reduce the viability of the cells of the cellular immunotherapeutic.

In a further embodiment, the present invention provides a method for minimising or reducing the risk of tumour lysis syndrome in a subject who has received or is receiving a therapy with a cellular immunotherapeutic for binding to a target antigen, the method comprising:
providing a subject who has received or is receiving a therapy with a cellular immunotherapeutic for binding to a target antigen on a cell;
administering to the subject, a polypeptide for binding to the cellular immunotherapeutic or a nucleic acid encoding said polypeptide; wherein the polypeptide comprises:
i) an epitope of the target antigen that competes for binding to the cellular immunotherapeutic, and the polypeptide thereby disrupts the interaction between the cellular immunotherapeutic and the target antigen on the cell;
ii) an amino acid sequence for triggering cell-mediated killing,
   wherein, upon binding of the polypeptide to the cellular therapeutic, the cellular immunotherapeutic is targeted for cell-mediated killing;
   thereby minimising or reducing the risk of tumour lysis syndrome in the subject.

In a further embodiment, the present invention provides a method for treating tumour lysis syndrome in a subject who has received or is receiving a therapy with a cellular immunotherapeutic for binding to a target antigen, the method comprising:
providing a subject who has received or is receiving a therapy with a cellular immunotherapeutic for binding to a target antigen on a cell;
administering to the subject, a polypeptide for binding to the cellular immunotherapeutic or a nucleic acid encoding said polypeptide; wherein the polypeptide comprises:
i) an epitope of a target antigen that competes for binding to the cellular immunotherapeutic, and the polypeptide thereby disrupts the interaction between the cellular immunotherapeutic and the target antigen on the cell;
ii) an amino acid sequence for triggering cell-mediated killing,
   wherein, upon binding of the polypeptide to the cellular therapeutic, the cellular immunotherapeutic is targeted for cell-mediated killing;
   thereby treating tumour lysis syndrome in the subject.

As used herein, tumour lysis syndrome refers to a group of metabolic abnormalities that can occur as a complication during the treatment of cancer, where large amounts of tumour cells are lysed at the same time by the treatment, releasing their contents into the bloodstream. This occurs most commonly after the treatment of lymphomas and leukaemias. In oncology and haematology, this is a potentially fatal complication, and patients at increased risk for TLS should be closely monitored before, during, and after their course of chemotherapy.

Tumour lysis syndrome is characterised by high blood potassium (hyperkalaemia), high blood phosphate (hyperphosphatasemia), low blood calcium (hypocalcaemia), high blood uric acid (hyperuricaemia), and higher than normal levels of blood urea nitrogen (BUN) and other nitrogen-containing compounds (azotaemia). These changes in blood electrolytes and metabolites are a result of the release of cellular contents of dying cells into the bloodstream from breakdown of cells. In this respect, TLS is analogous to rhabdomyolysis, with comparable mechanism and blood chemistry effects but with different cause. In TLS, the breakdown occurs after cytotoxic therapy or from cancers with high cell turnover and tumour proliferation rates. The metabolic abnormalities seen in tumour lysis syndrome can ultimately result in nausea and vomiting, but more seriously acute uric acid nephropathy, acute kidney failure, seizures, cardiac arrhythmias, and death

It will be appreciated that the methods of the present invention provide for flexibility in terms of the timing of the administration of the cellular immunotherapeutic and molecules as described herein. In certain embodiments of the first aspect of the invention, the timing of administration of the molecule (e.g., polypeptide) comprising an epitope of the target antigen may be such that the molecule is only administered upon first signs or indications that the activity of the cellular immunotherapeutic needs to be diminished (for example, evidence of an increased presence of circulating cytokines which may be predictive of an impending cytokine storm).

Alternatively, in accordance with the first aspect of the invention, the molecule may be administered after such time that the cellular immunotherapeutic has already successfully diminished a significant proportion of the tumour burden, but it is desired that the cellular immunotherapeutic be given an opportunity to repopulate/regain potency. In this way, the timing of administration of the molecule enables the clinician to temporarily turn off the cellular immunotherapy to prevent exhaustion of the cells. Moreover, it will be within the purview of the skilled person to determine an appropriate dose of molecule to administer, to enable a mild, moderate or significant reduction in the activity of the cells. Further still, the polypeptide can be administered according to a dosing gradient, to enable gradual dampening of the cell's activity.

Tonic activation of CAR T cells may lead to overactivation and activation-induced cell death. In the case of survival of the effector cells, tonic activation of CAR T cells via the CAR receptor leads to maturation to Teff cells with high expression of the markers PD-1, TIGIT, TIM-3 and others that are defined as markers of activation and lead to exhaustion of the immune cells. Exhausted cells are unable to undergo clonal expansion and they lose effector function. Chronic activation of T cells lead to their dysfunction and the same is true for CAR T cells. One major obstacle in cancer immunotherapy that recruits T cells is the chronic activation that can lead to: acute overactivation and activation induced cell death; and chronic tonic activation (without any period of reconvalescence) leading to dysfunction of T cells and CAR T cells. Providing cells with a rest or a period of recovery can restore a level of T cell function or CAR T cell function necessary for the long-term effector function of T cells and CAR T cells.

In contrast to so-called “liquid cancers”, clearance of solid cancer tumour cells by CAR T cells requires longer time intervals. In B-lineage malignancies like B cell precursor ALL treated by Kymriah (an anti-CD19 CAR T cell therapy), tumour clearance is found after 2 weeks in most patients and a complete response (complete MRD response) is found after 4 weeks at the latest. This means that all tumour cells are eliminated thus enabling the CAR T cells the chance to recover their function. In lymphoma, tumour clearance becomes more difficult as the time to complete response may take several months and T cells already show signs of dysfunction. The time of activation is non-physiologically prolonged. In solid cancer, tumour clearance may take even longer (greater than 6 months) and in combination with the tumour microenvironment, CAR T cells show dysfunction that is partially driven by the chronic tonic activation. Providing the cells with a periodic activation phase (functional phase of CARs) and a recovery phase (CARs do not recognise cancer cells during this time) facilitates their persistence.

As such, the methods of the first aspect of the invention may comprise a cycle of several weeks of treatment with a cellular immunotherapeutic (e.g., CAR T cell) prior to administration of the molecule for dampening the activity of the cell. The cycle may be a period of about 2 weeks, about 3 weeks, about 4 weeks, about 5 weeks, or about 6 weeks of activation prior to the administration of the molecule that promotes a reversible switching off or dampening of activity. The period of time in which activity is dampened or switched off may be a period of about 1 week, a period of about 2 weeks or longer. In certain embodiments, the period of CAR T activity is about 2 weeks, followed by about 2 weeks in which the CAR T activity is reversible inhibited, in accordance with the first aspect of the invention.

In accordance with the second aspect of the invention, it may be necessary in certain circumstances to rapidly clear and permanently switch off the activity of the cellular immunotherapeutic. Again, the timing of administration of the molecule will easily be determined by the clinician and may be upon signs of a potentially deleterious immune response triggered by the immunotherapeutic such that the activity of the cellular immunotherapeutic needs to be permanently switched off for the benefit of the subject receiving treatment.

### EXAMPLES

### Example 1: Blocking of Anti-nfP2X₇ CAR T Cell Killing

T cells were transduced with a lentiviral vector encoding an anti-nfP2X₇ CAR. The CAR used comprises an antigen binding domain for binding the E200 epitope of the receptor (as described elsewhere herein).

The CAR T cell product showed a CAR expression of 41%. The effector to target ratio was 5:1 (T cell to MOLM-13), which corresponds to an ET ratio of 2:1 (nfP2X7-CAR T to MOLM-13).

Cells from a MOLM-13 cell line that constitutively expressed firefly luciferase and eGFP reporters (MOLM-13_LUC_eGFP) were seeded at 25,000 cells per well plus the CAR T cells at the indicated ET.

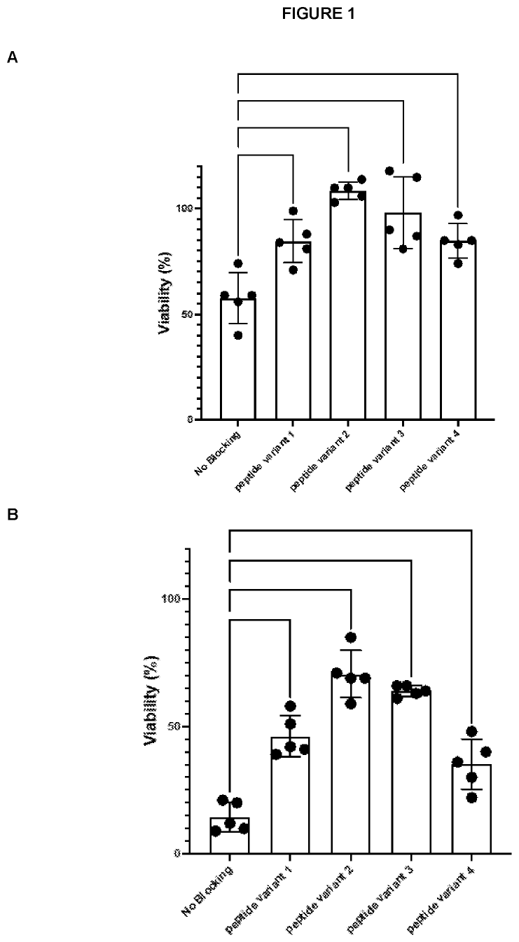

Four different peptide variants were used to reduce the killing of MOLM-13 cancer cells by the nfP2X₇ CAR T cells (FIG. 1A). All 4 peptides comprise the sequence of the E200 epitope of nfP2X₇, which is also the epitope bound by the nfP2X₇ CAR used in this study. The peptides comprise varying linker regions at the N- and C-terminal regions. The sequences of the peptides are provided in Table 1.

As shown in FIG. 1A, all 4 peptides were able to reduce the engagement of the direct antigen-binding CAR T cells to reduce the number of cancer cells indicated by the percentage of MOLM-13 cells that were still viable after 24 hours. This indicates a significant blocking of direct CAR function using peptides comprising the sequence of the epitope recognised by the CAR antigen binding domain.

A similar experiment was conducted using an “indirect” CAR T system. Briefly, MOLM-13 cells were co-cultured with anti-nfP2X₇ CAR T cells, along with a polypeptide comprising a) an epitope bound by the CAR T cells and b) an antigen binding domain for binding to CD33. In this way, the polypeptide enables binding of the anti-nfP2X₇ CAR T cells to MOLM-13 cells via a different antigen (being CD33). The further addition of peptide variants, as used in the first experiment, also significantly reduced the killing of MOLM-13 cells compared to when no peptides were added (FIG. 1B).

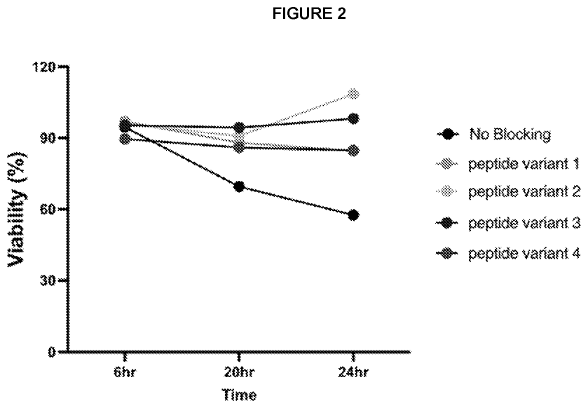

The kinetics of the reduction of the killing of MOLM-13 cancer cells by the nfP2X₇ CAR T cells are shown in FIG. 2. After 24 hours, the peptides continue to block cell killing by the CAR T cells whereas in the absence of the peptides, the CAR T cells continue to reduce MOLM-13 cell viability.

### Example 2: Blocking of Anti-nfP2X₇ CAR T Cell Killing is Due to Interaction of the Peptides with the nfP2X₇ CAR

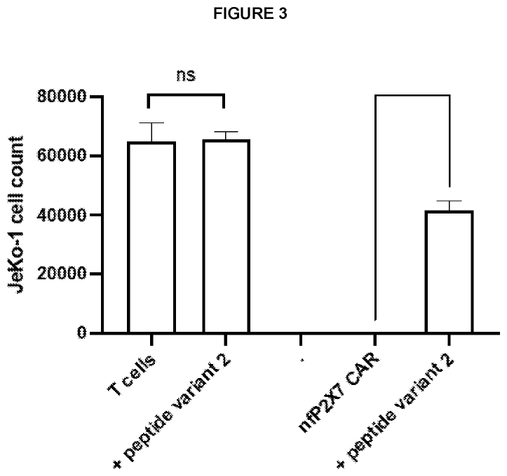

FIG. 3 shows that the reduction in cell killing by T cells is specifically due to the interaction of the peptides with the nfP2X₇ CAR. More specifically, T cells (either unmodified or expressing nfP2X₇ CAR) were cultured with JeKo-1 cells at an ET ratio of 5 to 1. Blocking was performed using peptide variant 2 at 5 μg/mL.

On day 5 of co-culture the cell counts were measured and evaluated for total JeKo-1 cell count. The total number of JeKo-1 cells added in the experiment was 20,000 on day 1 and day 3 of the experiment (therefore the total amount of added cells was 40,000 JeKo-1 per condition). In the first arm of the experiment, ie using T cells that do not express nfP2X₇ CAR, the JeKo-1 cells proliferated to 60,000 cells in total after 5 days in co-culture. This result was not changed by the addition of a blocking peptide.

In contrast, in the second arm of the experiment (ie using T cells expressing nfP2X₇ CAR), no JeKo-1 cells were observed on day 5 of co-culture when no blocking peptide was added. Addition of the blocking peptide variant 2 at 5 ug/mL reversed this such that after 5 days, 40,000 JeKo-1 cells were present in culture. These results show that killing of cancer cells by CAR T cells can be reduced (ie dampened) through the use of peptides that comprise an epitope that is recognised by the CAR expressed by the T cells.

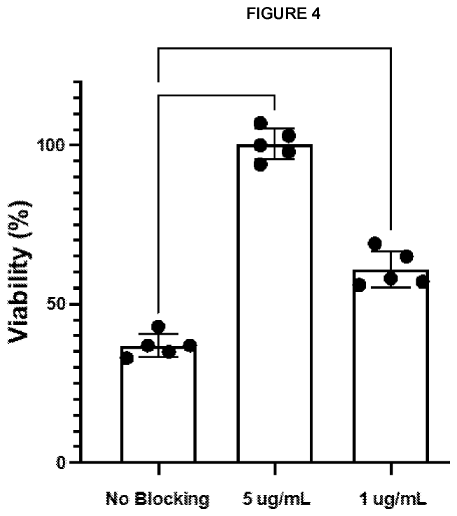

FIG. 4 also shows that the blocking of CAR T function due to the peptide can be controlled by reducing the amount of peptide used, with greater blocking of cell killing using greater amounts of peptide. In other words, the effect is dose-dependent.

It will be understood that the invention disclosed and defined in this specification extends to all alternative combinations of two or more of the individual features mentioned or evident from the text or drawings. All of these different combinations constitute various alternative aspects of the invention.

1. A method for inhibiting the activity of a cellular immunotherapeutic in a subject who has received or is receiving a therapy with a cellular immunotherapeutic for binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
wherein the epitope of the molecule competes with the epitope on the target antigen, for binding to the cellular immunotherapeutic and the molecule thereby disrupts the interaction between the cellular immunotherapeutic and the target antigen;
thereby inhibiting the activity of the cellular immunotherapeutic in the subject.

2. The method of claim 1, wherein the inhibition is reversible such that once the molecule has been cleared from the circulation of the subject, the activity of the cellular immunotherapeutic is restored.

3. The method of claim 2, wherein the method does not reduce the viability of the cells of the cellular immunotherapeutic.

4. A method for minimising or reducing the risk of an aberrant inflammatory response in a subject who has received or is receiving a therapy with a cellular immunotherapeutic or binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
wherein the epitope of the molecule competes with the epitope on the target antigen, for binding to the cellular immunotherapeutic and the molecule thereby disrupts the interaction between the cellular immunotherapeutic and the target antigen;
thereby inhibiting the activity of the cellular immunotherapeutic in the subject.

5. The method of claim 1, wherein the inhibition treats an aberrant inflammatory response in a subject who has received or is receiving a therapy with a cellular immunotherapeutic for binding to a target antigen.

6. The method of claim 4, wherein the aberrant inflammatory response comprises a cytokine-associated toxicity.

7. The method of claim 6, wherein the aberrant inflammatory responses comprises “cytokine release syndrome” (CRS) or hypercytokinemia.

8. A method for increasing the persistence of a cellular immunotherapeutic in a subject, wherein the cellular immunotherapeutic is for binding to a target antigen, the method comprising:
providing a subject who has received or is receiving a therapy with a cellular immunotherapeutic for binding to a target antigen;
administering to the subject, a molecule for binding to the cellular immunotherapeutic, the molecule comprising or consisting of an epitope of the target antigen;
wherein the epitope of the molecule competes with the epitope of the target antigen, for binding to the cellular immunotherapeutic and the molecule thereby disrupts the interaction between the cellular immunotherapeutic and the target antigen;

9. (canceled)

10. The method of claim 1, wherein the target antigen is expressed or present on the surface of a cancer cell.

11. The method of claim 1, wherein the molecule comprises a fusion protein that comprises the epitope, and a further sequence for facilitating improved solubility and stability of the polypeptide.

12. The method of claim 11, wherein the further sequence comprises an Fc region of an antibody, wherein the Fc region of an antibody does not trigger ADCC or CDC.

13. (canceled)

14. The method of claim 1, wherein the molecule further comprises
a compound for triggering cell death.

15.-16. (canceled)

17. The method of claim 14, wherein the molecule reduces the viability of the cells of the cellular immunotherapeutic.

18.-20. (canceled)

21. The method of claim 14, wherein the molecule is in the form of a fusion protein comprising the epitope and a further sequence for facilitating targeting of the cellular immunotherapeutic for cell-mediated killing by cytotoxic immune cells, when the cellular immunotherapeutic is bound by the polypeptide.

22. The method of claim 21, wherein the further sequence comprises an Fc region of an antibody.

23. The method of claim 22, wherein the Fc region exhibits effector function and binds the Fcγ Receptor (FCγR) triggering ADCC or CDC.

24. The method of claim 1, wherein the cellular immunotherapeutic comprises an immune cell, or progenitor thereof, wherein the immune cell expresses a receptor comprising an antigen-recognition domain and a signalling domain.

25. The method of claim 24, wherein the antigen-recognition domain binds to a tumour-specific or tumour-associated antigen expressed on a cell surface.

26. (canceled)

27. The method of claim 25, wherein the antigen-recognition domain binds to a tumour-associated antigen selected from the group consisting of: CD33 (Siglec-3), CD123 (IL3RA), CD135 (FLT-3), CD44 (HCAM), CD44V6, CD47, CD184 (CXCR4), CLEC12A (CLL1), FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, PCSA, CD276, TROP-2, DLL1, PSMA, and dysfunctional (nf)P2X₇ receptor

28. The method of claim 27, wherein the antigen-recognition domain of the cellular immunotherapeutic binds to dysfunctional P2X₇ receptor.

29.-42. (canceled)

43. The method of claim 17, wherein the molecule targets the cells for degradation/killing.

44. The method of claim 21, wherein the cytotoxic immune cells are cytotoxic T-lymphocytes or NK cells.
